(19) United States
(12) Patent Application Publication
(10) Pub. No.: US 2025/0034282 A1
(43) Pub. Date: Jan. 30, 2025

(54) ARRDC1-Mediated Microvesicles (ARMMS) Degrading System and Uses Thereof

(71) Applicant: Vesigen, Inc., Cambridge, MA (US)

(72) Inventors: Robert Millman, Cambridge, MA (US); Joseph Nabhan, Cambridge, MA (US); Nicholas Buffinger, Cambridge, MA (US)

(21) Appl. No.: 18/717,882

(22) PCT Filed: Dec. 8, 2022
(86) PCT No.: PCT/US2022/081213

§ 371 Date: Jun. 7, 2024

Related U.S. Application Data
(60) Provisional application No. 63/287,683, filed on Dec. 9, 2021.

(51) Int. Cl. C07K 16/44; C07K 14/005; C07K 14/435

(52) U.S. Cl. CPC C07K 16/44; C07K 14/005; C07K 14/435; C12N 2740/16022

(57) ABSTRACT

This invention relates generally to ARRDC1 fusion proteins, ARRDC1-mediated microvesicles (ARMMs) degrading systems, and their uses.

### RELATED CASES

This application is entitled to and claims the benefit of the filing date of U.S. provisional application U.S. 63/287,683, filed Dec. 9, 2021.

### BACKGROUND

Protein degradation is a highly regulated process that maintains cellular homeostasis. The ubiquitin-proteasome pathway (UPP) is a common way of protein degradation in eukaryotic cells. Numerous cellular pathways are regulated by the UPP mechanism, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection. Degradation of a protein via the UPP involves two steps: first, tagging of the substrate by covalent attachment of multiple ubiquitin molecules and second, degradation of the tagged protein by the 26S proteasome complex with release of free and reusable ubiquitin. The E3 ubiquitin ligase plays a critical role in the UPP, determining the substrate specificity and covalently attaching multiple ubiquitin molecules to a terminal lysine residue of the substrate protein. The UPP allows decrease of the expression of cellular proteins without the need for genomic modification. There is a need for the development of new systems for targeted protein degradation via the UPP.

### SUMMARY

Provided herein are arrestin domain-containing protein 1 (ARRDC1)-mediated microvesicles (ARMMs) comprising degrading systems and their uses. Particularly, provided herein is an ARRDC1 fusion proteins comprising: an ARRDC1 protein or a variant thereof, and a non-enzymatic targeting domain that recruits a target protein. In some embodiments, the targeting domain is fused to the N-terminal end of the ARRDC1 protein or variant thereof. In some embodiments, the targeting domain is fused to the C-terminal end of the ARRDC1 protein or variant thereof. In some embodiments, the ARRDC1 protein or variant thereof is fused to the targeting domain by a linker. In some embodiments, the ARRDC1 protein or variant thereof comprises at least two PPXY motifs. In some embodiments, the at least two PPXY motifs are different. In some embodiments, the non-enzymatic targeting domain comprises an interaction domain. In some embodiments, the interaction domain comprises a non-enzymatic protein binding peptide. In some embodiments, the target protein comprises: a transcription factor, a tumor promoter or the protein product of an oncogene, a developmental regulator, a growth factor, a metastasis promoter, an anti-apoptotic protein, a membrane-associated protein, a transmembrane receptor, an enzyme, a nuclease, a recombinase, or a reprogramming factor. In some embodiments, the non-enzymatic targeting domain comprises an intrabody. In some embodiments, the non-enzymatic targeting domain comprises a single domain antibody (sdAb). In some embodiments the sdAb is a camelid single domain. In some embodiments, a terminal of the non-enzymatic targeting domain is fused to at least one additional PPXY motif. In some embodiments, the terminal of the non-enzymatic targeting domain is fused to at least two additional PPXY motifs. In some embodiments, the at least two additional PPXY motifs are different PPXY motifs. In some embodiments, the non-enzymatic targeting domain is fused to a Fc region of an antibody. In some embodiments, the non-enzymatic targeting domain or the Fc region of an antibody is fused to an RNA binding protein. In some embodiments, the RNA binding protein is a tat protein. In some embodiments, the tat protein is an HIV tat protein or a bovine immunodeficiency virus (BIV) tat protein. In some embodiments, the targeting domain comprises a bound aptamer. In some embodiments, the targeting domain is fused to a ubiquitin ligase. In some embodiments, the linker is a cleavable linker. In some embodiments, the cleavable linker comprises a protease cleavage site. In some embodiments, the protease cleavage site comprises a furin cleavage site, a viral protease cleavage site, or other cleavage site recognized by a viral protease. In some embodiemtns, the viral protease cleave site comprises an HIV-1 protease cleavage site or a tobacco etch virus (TEV) cleavage site. In some embodiments the cleavable linker is a photocleavable linker that comprises a photocleavable protein or fragment thereof. In some embodiments the photocleavable linker comprises the photocleavable protein PhoCl1 (Zhang et al., Nat. Methods 2017 April; 14(4):391-394; doi: 10.1038/nmeth.4222) or a fragment thereof. In some embodiments the photocleavable linker comprises a protein or protein fragment derived from PhoCl1, such as PhoCl2 (Lu et al., Chem. Sci., 2021 May 31; 12(28):9658-9672; doi: 10.1039/d1sc01059j). In some embodiments, the ubiquitin ligase is a ubiquibody. In some embodiments, the ubiquibody is CHIP (carboxyl terminus of Hsc70-interacting protein) or a fragment thereof.

In some embodiments, the non-enzymatic targeting domain of the ARRDC1 fusion protein comprises a ubiquitination-specific antibody domain. In some embodiments, the ARRDC1 fusion protein comprises on or more FLAG-tags. In some embodiments, the one or more of these FLAG-tags comprise a sequence of DYKDDDK (SEQ ID NO: 90). The present invention is not limited to compsitions comprising FLAG-tags, indeed, additional compostions comprise one or more affinity tags (e.g., GST, His, CBP, MBP, and the like), and epitope tags (e.g., Myc, HA, and the like).

Further provided herein is an ARRDC1-mediated microvesicle (ARMM), comprising: (i) a lipid bilayer; and (ii) the ARRDC1 fusion protein or a variant thereof as disclosed herein. In some embodiments, the ARMM further comprises a fusion RNA, wherein the fusion RNA comprises (i) an RNA sequence that binds the RNA binding protein and (ii) an expression sequence encoding a ubiquitin ligase. In some embodiments, the RNA sequence is a TAR sequence. In some embodiments, the ubiquitin ligase is Trim21.

Further provided herein is a nucleic acid construct encoding the ARRDC1 fusion protein as disclosed herein.

Further provided herein is a microvesicle-producing cell, comprising a recombinant expression construct encoding the ARRDC1 fusion protein as disclosed herein under the control of a heterologous promoter. In some embodiments, the microvesicle-producing cell further comprises a recombinant expression construct encoding a fusion RNA under the control of a heterologous promoter, wherein the fusion RNA comprises (i) an RNA sequence that binds the RNA binding protein and (ii) an expression sequence encoding a ubiquitin ligase. In some embodiments, the RNA sequence is a TAR sequence. In some embodiments, the ubiquitin ligase is Trim21.

Further provided herein is a method of degrading a target protein, comprising contacting the target cell with the microvesicle as disclosed herein.

Further provided herein is a method of altering expression of at least one gene, comprising contacting the target cell with the microvesicle as disclosed herein. In some embodiments, the expression of at least one gene is decreased. In some embodiments, the expression of at least one gene is increased.

Further provided is a method of preparing an arrestin domain-containing protein 1 (ARRDC1)-mediated microvesicle (ARMM), comprising (a) culturing a host cell comprising a recombinant expression construct encoding the ARRDC1 fusion protein under the control of a heterologous promoter in a medium: and (b) collecting the ARMMs from the host cell or the medium. In some embodiments, the host cell has a reduced expression level of ubiquitin ligase as compared to a host cell of wildtype. In some embodiments, the host cell lacks the ubiquitin ligase. In some embodiments, the host cell has a reduced expression level of said target protein as compared to a host cell of wildtype. In some embodiments, the host cell lacks said target protein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention. there are shown in the drawings embodiments, which are presently exemplified. It should be understood, however, that the invention is not limited to the precise arrangement and instrumentalities of the embodiments shown in the drawings.

FIG. 1A illustrates a non-limiting schematic representation of the fusion protein used for targeted protein degradation. Specifically, an ARRDC1 protein, containing two PPXY (SEQ ID NO: 2) motifs, is fused to a polypeptide targeting domain. FIG. 1B illustrates an ARRDC1-RNA binding protein fusion, containing a bound aptamer as the targeting domain.

FIG. 2A is a schematic demonstrating a fusion protein used for targeted protein degradation. Specifically, an ARRDC1 protein, containing two PPXY (SEQ ID NO: 2) motifs, is fused to an anti-mCherry single domain antibody (sdAb). The anti-mCherry sdAb specifically recruits and binds a mCherry fluorescent protein.

FIG. 2B provides an immunofluorescence image showing that 293T cells co-transfected with the fusion protein expression construct of FIG. 2A and an mCherry expression construct had a decreased mCherry protein level.

FIG. 2C provides Western blots showing that 293T cells co-transfected with the fusion protein expression construct of FIG. 2A and an mCherry expression construct had decreased mCherry protein levels in a dose-dependent manner.

FIG. 3 illustrates a non-limiting schematic representation of the fusion protein used for targeted protein degradation. Specifically, an ARRDC1 protein, containing two PPXY (SEQ ID NO: 2) motifs, is fused to a targeting domain whose N-terminal end is associated with two additional PPXY (SEQ ID NO: 2) motifs.

FIG. 4 illustrates a non-limiting schematic representation of the fusion protein used for targeted protein degradation. Specifically, an ARRDC1 protein, containing two PPXY (SEQ ID NO: 2) motifs, is fused to a targeting domain fused to Fc (fragment crystallizable) region of an antibody. The C-terminal end of the targeting domain is further associated with a tat protein. The tat protein is able to recruit a TAR-Trim21 fusion RNA. Trim21 is able to ubiquitnate and degrade Fc bound proteins.

FIG. 5 illustrates a non-limiting schematic representation of the fusion protein used for targeted protein degradation. Specifically, an ARRDC1 protein, containing two PPXY (SEQ ID NO: 2) motifs, is fused to a targeting domain fused to a ubiquibody such as CHIP.

FIG. 6 illustrates a non-limiting schematic representation of the fusion protein used for targeted protein degradation. Specifically, a protease cleavage site exists between the ARRDC1 protein and the target recruitment/degrader module as described above.

FIG. 7 illustrates a non-limiting schematic representation of the fusion protein used for targeted protein degradation, illustrates a non-limiting schematic representation of the fusion protein used for targeted protein degradation. Specifically, an ARRDC1 protein, containing two PPXY (SEQ ID NO: 2) motifs fused to a targeting domain fused to Fc (fragment crystallizable) region of an antibody, and a protease cleavage site exists between the ARRDC1 protein and the target recruitment/degrader module as described above.

FIG. 8 demonstrates the results of western blots stained with anti-GAPDH (gel/blot loading control), ant-ARRDC1, and anti-mCherry. Negative controls are provided in lanes 1 and 5 of the western blots. As shown in FIG. 8, target cell mCherry levels were unaffected by negative controls. but reduced by ARMMs-delivered ARRDC1/anti-mCherry sdAb fusion protein, particularly the VSV-G pseudotyped ARMMs particles.

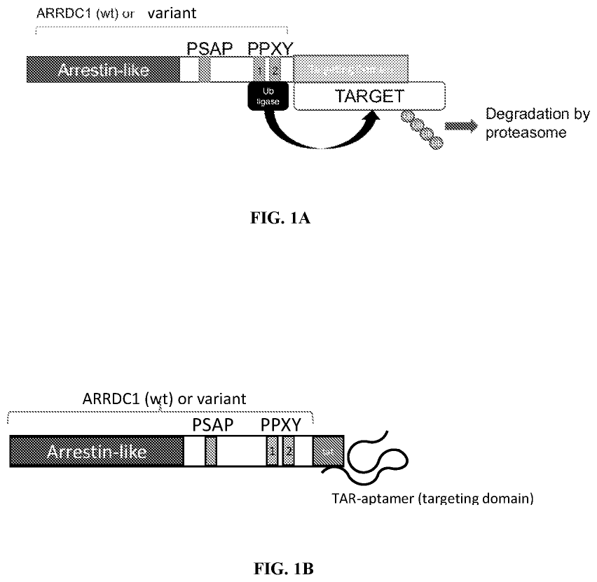

### DETAILED DESCRIPTION

This disclosure relates generally to ARRDC1-mediated microvesicles (ARMMs) degrading systems and their uses. Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### Definitions

As used herein. “antibody” refers to immunoglobulins and immunoglobulin fragments, whether natural or partially or wholly synthetically, such as recombinantly, produced, including any fragment thereof containing at least a portion of the variable region of the immunoglobulin molecule that retains the binding specificity ability of the full-length immunoglobulin. Hence, an antibody includes any protein having a binding domain that is homologous or substantially homologous to an immunoglobulin antigen-binding domain (antibody combining site). Antibodies also include single domain antibodies (sdAb). Such sdAbs may be isolated from members of the Camelidae family of mammals, including llamas, camels, and alpacas. Single domain antibodies can comprise a heavy chain variable domain (VH) of a peptide, ligand, or small molecule. Single domain antibodies can comprise a variable domain (VHH) of camelid derived sdAb, peptide, ligand or small molecule. Antibodies can include sdAbs which recognize tissue-specific markers, such as is disclosed in U.S. Pat. No. 9,816,080. Such sdAbs for use in the constructs provided herein can be selected via phage display libraries using methods known in the art (see, e.g., Velazquez E J, Cress J D, Humpherys T B, Mortimer T O, Bellini D M, Skidmore J R, et al. (2022) Selection of human single domain antibodies (sdAb) against thymidine kinase I and their incorporation into sdAb-Fc antibody constructs for potential use in cancer therapy. PLoS ONE17 (3): e0264822, https://doi.org/10.1371/journal.pone.0264822). Antibodies (e.g., sdAbs) which promote ubiquitination (e.g., ubiquitination-specific antibodies) may be incorporated into the constructs provided herein. Antibodies include antibody fragments. As used herein, the term “antibody,” thus, includes synthetic antibodies, recombinantly produced antibodies, multispecific antibodies (e.g., bispecific antibodies), human antibodies, non-human antibodies, humanized antibodies, chimeric antibodies, intrabodies, and antibody fragments, such as, but not limited to, Fab fragments, Fab′ fragments, F(ab′)2 fragments, Fv fragments, disulfide-linked Fvs (dsFv). Fd fragments, Fd′ fragments, single-chain Fvs (scFv), single-chain Fabs (scFab), diabodies, anti-idiotypic (anti-Id) antibodies, or antigen-binding fragments of any of the above. Antibodies provided herein include members of any immunoglobulin type (e.g., IgG, IgM, IgD, IgE, IgA and IgY), any class (e.g. IgG1, IgG2, IgG3, IgG4, IgA1 and IgA2) or subclass (e.g., IgG2a and IgG2b).

As used herein, an “antibody fragment” or “antigen-binding fragment” of an antibody refers to any portion of a full-length antibody that is less than full length but contains at least a portion of the variable region of the antibody that binds antigen (e.g., one or more CDRs and/or one or more antibody combining sites) and thus retains the binding specificity, and at least a portion of the specific binding ability of the full-length antibody. Hence, an antigen-binding fragment refers to an antibody fragment that contains an antigen-binding portion that binds to the same antigen as the antibody from which the antibody fragment is derived. Antibody fragments include antibody derivatives produced by enzymatic treatment of full-length antibodies, as well as synthetically, e.g., recombinantly produced derivatives. An antibody fragment is included among antibodies. Examples of antibody fragments include, but are not limited to, single-domain antibody (sdAb), Fab, Fab′, F(ab′)2, single-chain Fv (scFv), Fv, dsFv, diabody, Fd and Fd′ fragments and other fragments, including modified fragments (see, for example, Methods in Molecular Biology, Vol 207: Recombinant Antibodies for Cancer Therapy Methods and Protocols (2003); Chapter 1; p 3-25, Kipriyanov). The fragment can include multiple chains linked together, such as by disulfide bridges and/or by peptide linkers. An antibody fragment generally contains at least or about 50 amino acids and typically at least or about 200 amino acids. An antigen-binding fragment includes any antibody fragment that when inserted into an antibody framework (such as by replacing a corresponding region) results in an antibody that immunospecifically binds (i.e., exhibits Ka of at least or at least about 107-108 M-1) to the antigen.

As used herein, the term “aptamer” refers to nucleic acids that bind to a specific target molecule, e.g., an RNA binding protein. In some embodiments, nucleic acid (e.g., DNA or RNA) aptamers arc engineered through repeated rounds of in vitro selection or equivalently, SELEX (systematic evolution of ligands by exponential enrichment) methodology to bind to various molecular targets, for example, proteins, small molecules, macromolecules, metabolites, carbohydrates, metals, nucleic acids, cells, tissues, and organisms. Methods for engineering aptamers to bind to various molecular targets, such as proteins, are known in the art and include those described in U.S. Pat. Nos. 6,376,19; and 9,061,043; Shui B., et al., “RNA aptamers that functionally interact with green fluorescent protein and its derivatives.” Nucleic Acids Res., March; 40(5): e39 (2012); Trujillo U. H., et al., “DNA and RNA aptamers: from tools for basic research towards therapeutic applications.” Comb Chem High Throughput Screen 9 (8): 619-32 (2006); Srisawat C., et al., “Streptavidin aptamers: Affinity tags for the study of RNAs and ribonucleoproteins.” RNA, 7:632-641 (2001); and Tuerk and Gold, “Systematic evolution of ligands by exponential enrichment: RNA ligands to bacteriophage T4 DNA polymerase.” Science. 1990; the entire contents of each of which are hereby incorporated by reference in their entirety.

As used herein, the term “ARMM” refers to a microveside comprising an ARRDC1 protein or a variant thereof. The molecular mechanism by which ARMMs are produced is described in U.S. Pat. No. 10,945,954B, which is incorporated by reference herein. In some embodiments, the ARMM is shed from a cell, and comprises a molecule, for example, a nucleic acid, protein, or small molecule, present in the cytoplasm of the cell. In some embodiments, the ARMM is shed from a transgenic cell comprising a recombinant expression construct that includes the transgene, and the ARMM comprises a gene product, for example, a transcript or a protein encoded by the expression construct. In some embodiments, the ARMM is produced synthetically, for example, by contacting a lipid bilayer within ARRDC1 protein in a cell free system in the presence of TSG101. HECT domain ligase, and VPS4a. In some embodiments, an ARMM lacks a late endosomal marker. Some ARMMs as provided herein do not include, or are negative for, one or more exosomal biomarker. Exosomal biomarkers are known to those of skill in the art and include, but are not limited to CD63, Lamp-1, Lamp-2, CD9, HSPA8, GAPDH, CD81, SDCBP, PDCD6IP, ENOi, ANXA2, ACTB, YWHAZ, HSP90AAi, ANXAS, EEFiAi, YWHAE, PPIA, MSN, CFL1, ALDOA, PGKi, EEF2, ANXA1, PKM2, HLA-DRA, and YWHAB. For example, some ARMMs provided herein lack CD63, some ARMMs lack LAMP 1, some ARMMs lack CD9, some ARMMs lack CD81, some ARMMs lack CD63 and Lamp 1, some ARMMs lack CD63, Lamp 1, and CD9, some ARMMs lack CD63, Lamp-1, CD81 and CD9, and so forth. Certain ARMMs provided herein may include an exosomal biomarker. Accordingly, some ARMMs may be negative for one or more exosomal biomarker, but positive for one or more different exosomal biomarker. For example, such an ARMM may be negative for CD63 and Lamp-1, but may include PGKi or GAPDH; or may be negative for CD63, Lamp-1, CD9 and CD81, but may be positive for HLA-DRA. In some embodiments, ARMMs include an exosomal biomarker, but at a lower level than a level found in exosomes. For example, some ARMMs include one or more exosomal biomarkers at a level of less than about 1%, less than about 5%, less than about 10%, less than about 20%, less than about 30%, less than about 40%, or less than about 50% of the level of that biomarker found in exosomes. To give a non-limiting example, in some embodiments, an ARMM may be negative for CD63 and Lamp-1, include CD9 at a level of less than about 5% of the level of CD9 typically found in exosomes, and be positive for ACTB. Exosomal biomarkers in addition to those listed above are known to those of skill in the art, and the invention is not limited in this regard.

As used herein, the term “associated with,” when used with respect to two or more molecular entities, for example, fusion proteins, molecules, nucleic acids, means that the entities are physically associated or connected with one another, either directly or via one or more additional moieties that services as a linker, to form a structure that is sufficiently stable so that the entities remain physically associated under the conditions in which the structure is used, e.g., physiological conditions. In some embodiments, the association is via a linker, for example, a cleavable linker.

As used herein, the term “binding RNA” refers to a ribonucleic acid (RNA) that binds to an RNA binding protein, for example, any of the RNA binding proteins known in the art and/or provided herein. In some embodiments, a binding RNA is an RNA that specifically binds to an RNA binding protein. A binding RNA that “specifically binds” to an RNA binding protein, binds to the RNA binding protein with greater affinity, avidity, more readily, and/or with greater duration than it binds to another protein, such as a protein that does not bind the RNA or a protein that weakly binds to the binding RNA. In some embodiments, the binding RNA is a naturally-occurring RNA, or non-naturally-occurring variant thereof, that binds to a specific RNA binding protein. For example, the binding RNA may be a TAR element, a Rev response element, an MS2 RNA, or any variant thereof that specifically binds an RNA binding protein. In some embodiments, the binding RNA may be a trans-activating response element (TAR element), or variant thereof, which is an RNA stem-loop structure that is found at the 5′ ends of nascent HIV-1 transcripts and specifically binds to the trans-activator of transcription (Tat) protein. In some embodiments, the binding RNA is a Rev response element (RRE), or variant thereof, that specifically binds to the accessory protein Rev (e.g., Rev from HIV-1). In some embodiments, the binding RNA is an MS2 RNA that specifically binds to a MS2 phage coat protein.

As used herein, the term “bioactive agent” is used to describe an agent, other than an ARMM or an ARRDC1 fusion protein according to the present invention, which is used in combination or alternation with the ARMM or the ARRDC1 fusion protein as an agent with biological activity to assist in effecting an intended or desired result of therapy, inhibition and/or prevention/prophylaxis for which the present compounds are used. Bioactive agents for use herein may include agents which have pharmacological activity similar to that for which the ARMM or the ARRDC1 fusion protein are used or administered and include for example, anti-cancer agents, antiviral agents, antimicrobial agents, antifungal agents, etc.

A “derivative” includes a polypeptide or fragment thereof having conservative amino acid substitutions relative to a second polypeptide; or a polypeptide or fragment thereof that is modified by covalent attachment of a second molecule such as, e.g., by attachment of a heterologous polypeptide, or by glycosylation, acetylation, phosphorylation, and the like. For example, polypeptides containing one or more analogs of an amino acid (e.g., unnatural amino acids and the like), polypeptides with unsubstituted linkages, as well as other modifications known in the art, both naturally and non-naturally occurring.

As used herein, the term “domain” refers to a folded protein structure which retains its tertiary structure independent of the rest of the protein. Generally, domains are responsible for discrete functional properties of proteins and in many cases, may be added, removed or transferred to other proteins without loss of function of the remainder of the protein and/or of the domain.

As used herein. “expression” of a nucleic acid sequence refers to one or more of the following events: (1) production of an RNA transcript from a DNA sequence (e.g., by transcription); (2) processing of an RNA transcript (e.g., by splicing, editing, 5′ cap formation, and/or 3′ end processing); (3) translation of an RNA transcript into a polypeptide or protein; and (4) post-translational modification of a polypeptide or protein.

As used herein, the term “fragments” refer to fragments of the full, wild-type sequences which still retain the binding function of the wild type protein from which they are derived (e.g. fragments of the target protein still enable binding to the compound which mediates the interaction with the ubiquitin ligase). Fragments may suitably comprise at least 10 amino acids in length, for example 25, 50, 75, 80, 90, 100, 110, 120 or 130 amino acids in length. Fragments may also comprise a C-terminal truncation, or an N-terminal truncation.

As used herein, the term “fusion protein” generally refers to a protein translated from a fusion gene, which is created by joining parts of two different genes/nucleic acid sequences. The ARRDC1 or a variant thereof and the non-enzymatic targeting domain are associated with one another. preferably by genetic fusion (i.e. the fusion protein is generated by translation of a nucleic acid in which a polynucleotide encoding all or a portion of ARRDC1 is joined in-frame with a polynucleotide encoding all or a portion of the non-enzymatic targeting domain) or chemical conjugation to one another.

As used herein, the term “fusion RNA” generally refers to an RNA transcribed from a fusion gene, which is created by joining parts of two different genes/nucleic acid sequences. In the present disclosure, the RNA sequence that binds an RNA binding protein is associated with an expression sequence of a ubituitin ligase (such as Trim21 mRNA), by genetic fusion or chemical conjugation to one another.

As used herein, the term “interaction domain” refers to peptides or proteins which are adapted to specifically interact with target regions (or targets) on other molecules differing from themselves.

As used herein, the term “intrabody” refers to a form of antibody that is not secreted from a cell in which it is produced, but instead target one or more intracellular protein. Intrabodies may be used to affect a multitude of cellular processes including, but not limited to intracellular trafficking, transcription, translation, metabolic processes, proliferative signaling and cell division. Intrabodies are generally expressed within a cell via delivery of an expression cassette encoding the antibody, typically as a scFv, and comprising various localization signals to target the antibody to an intracellular compartment of interest (see Lo et al. (2008) Handb Exp Pharmacol 181:343-373, which is incorporated herein in its entirety). Methods of stabilizing intrabodies are known in the art and include, but are not limited to modifications of immunoglobulin VL domains that lead to hyperstability (Cohen (1998) Oncogene 17(19):2445-2456) or expression of the antibodies as a fusion protein to other stable intracellular proteins, such as maltose binding protein (Shaki-Loewenstein (2005) J Immunol Methods 303(1-2):19-39.

As used herein, the term “linker” refers to a chemical moiety linking two molecules or moieties, e.g., an ARRDC 1 protein and a targeting domain, or a targeting domain and a Tat protein. Typically, the linker is positioned between, or flanked by, two groups, molecules, or other moieties and connected to each one via a covalent bond, thus connecting the two. In some embodiments, the linker comprises an amino acid or a plurality of amino acids (e.g., a peptide or protein). In some embodiments, the linker comprises a nucleotide (e.g., DNA or RNA) or a plurality of nucleotides (e.g., a nucleic acid). In some embodiments, the linker is an organic molecule, group, polymer, or other chemical moiety. In some embodiments, the linker is a cleavable linker, e.g., the linker comprises a bond that can be cleaved upon exposure to, for example, UV light or a hydrolytic enzyme, such as a lysosomal protease. In some embodiments, the linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids). In other embodiments, the linker is a chemical bond (e.g., a covalent bond).

As used herein, the term “linker peptide” refers to a peptide used for association with at least one other peptide or protein, preferably in the form of a fusion protein.

As used herein, the term “microvesicle” refers to a droplet of liquid surrounded by a lipid bilayer. In some embodiments, a microvesicle has a diameter of about 10 nm to about 1000 nm. In some embodiments, a microvesicle has a diameter of at least about 10 nm, at least about 20 nm, at least about 30 nm, at least about 40 nm, at least about 50 nm, at least about 60 nm, at least about 70 nm, at least about 80 nm, at least about 90 nm, at least about 100 nm, at least about 125 nm, at least about 150 nm, at least about 175 nm, at least about 200 nm, at least about 250 nm, at least about 300 nm, at least about 400 nm, or at least about 500 nm. In some embodiments, a microvesicle has a diameter of less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 90 nm, less than about 80 nm, less than about 70 nm, less than about 60 nm, or less than about 50 nm. In some embodiments, the microvesicle have a diameter of about 50 to about 70 nm, as measured by nano-scale flow analysis (e.g., using the Flow Nanoanalyzer produced by NanoFCM) or transmission electron miscroscopy (TEM). In some embodiments the microvesicles have a diameter of about 70 to about 110 nm as measured by nanoparticle tracking analysis (NTA). In some embodiments, the microvesicles have a diameter of about 70 to about 90 nm as measured by tunable resistive pulse sensing (TRPS). In some embodiments, the microvesicles have a diameter of about 70 to about 90 nm as measured by tunable resistive pulse sensing (TRPS). The term “microvesicle” includes microvesicle shed from cells as well as synthetically produced microvesicles. Microvesicles shed from cells typically comprise the antigenic content of the cells from which they originate. Microvesicles shed from cells also typically comprise an asymmetric distribution of phospholipids, reflecting the phospholipid distribution of the cells from which they originate. In some embodiments, the inner membrane of microvesicles provided herein, e.g., of some ARMMs, comprises the majority of aminophospholipids, phosphatidylserine, and/or phosphatidylethanolamine within the lipid bilayer.

As used herein, the term “neoplasia” or “cancer” refers to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

As used herein, the term “non-enzymatic targeting domain” generally refers to a targeting domain that does not comprise an enzyme or enzymatically active fragment thereof.

As used herein, the term “recruit” refers to the attraction of entities such as a protein or an RNA molecule to the targeting domain or the RNA binding domain. The entities may bind to the targeting domain or the RNA binding domain.

As used herein, the term “reprogramming factor” refers to a factor that, alone or in combination with other factors, can change the state of a cell from a somatic, differentiated state into a pluripotent stem cell state. Non-limiting examples of reprogramming factors include a protein (e.g., a transcription factor), a peptide, a nucleic acid, or a small molecule. Known reprogramming factors that are useful for cell reprogramming include, but are not limited to Oct4, Sox2, Klf4, and c-myc. Similarly, a programming factor may be used to modulate cell differentiation, for example, to facilitate or induce cell differentiation towards a desired lineage.

As used herein, the term “RNA binding protein” refers to a polypeptide molecule that binds to a binding RNA, for example, any of the binding RNAs known in the art and/or provided herein. In some embodiments, an RNA binding protein is a protein that specifically binds to a binding RNA. An RNA binding protein that “specifically binds” to a binding RNA, binds to the binding RNA with greater affinity, avidity, more readily, and/or with greater duration than it binds to another RNA, such as a control RNA (e.g., an RNA having a random nucleic acid sequence) or an RNA that weakly binds to the RNA binding protein. In some embodiments, the RNA binding protein is a naturally-occurring protein, or non-naturally-occurring variant thereof, that binds to a specific RNA. For example, in some embodiments, the RNA binding protein may be a trans-activator of transcription (Tat) protein that specifically binds a trans-activating response element (TAR element). In some embodiments, the RNA binding protein is a regulator of virion expression (Rev) protein (e.g., Rev from HIV-1) or variant thereof, that specifically binds to a Rev response element (RRE). In some embodiments, the RNA binding protein is a coat protein of an MS2 bacteriophage that specifically binds to an MS2 RNA. The RNA binding proteins useful in the present disclosure (e.g., a binding protein fused to ARRDC1) may be designed to specifically bind a binding RNA (e.g., a binding RNA fused to a mRNA encoding a ubiquitin ligase such as Trim21) in order to facilitate ubiquitination of a target protein.

As used herein, the term “subject” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, the term “target protein” generally refers to a protein that is targeted and recruited to the non-enzymatic targeting domain. For example, the target protein may be an antigen or ligand. The target protein may be a transcription factor, a tumor promoter or the protein product of an oncogene, a developmental regulator, a growth factor, a metastasis promoter, an anti-apoptotic protein, a membrane-associated protein, a transmembrane receptor, an enzyme, a nuclease, a recombinase, or a reprogramming factor. In some embodiments, the ARRDC1 fusion proteins and the ARMMs disclosed herein are engineered to bring a ubiquitin ligase into proximity with a target protein, promoting ubiquitination of the target protein and ultimately its targeted degradation by the proteosome pathway.

As used herein, the term “targeting domain” refers to an oligomer or polymer composed of nucleic acid or amino acid subunits that is capable of binding a specific target, such as an antigen or ligand. The targeting domain may be, for example, any type of aptamer or protein which binds to a target protein.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to a disease, disorder, and/or condition, to treat, improve symptoms of, diagnose, prevent, and/or delay the onset of the disease, disorder, and/or condition.

As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

As used herein, the term “treating” refers to partially or completely alleviating, ameliorating, improving, relieving, delaying onset of, inhibiting progression of, reducing severity of, and/or reducing incidence of one or more symptoms or features of a particular disease, disorder, and/or condition. For example. “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “ubiquibody” generally refers to a protein chimera that combines the activity of E3 ubiquitin ligases with designer binding proteins to recruit target protein to the UPP for degradation. For example, a modular human E3 ubiquitin ligase CHIP (carboxyl terminus of Hsc70-interacting protein) whose natural substrate-binding domain is replaced with a single-chain Fv (scFv) intrabody or a fibronectin type III domain monobody that target their respective antigens with high specificity and affinity. Additional information of ubiquibodies can be found in Alyse Portnoff, Erin Stephens, Jeffrey Varner, et al., “Ubiquibodies, Synthetic E3 Ubiquitin Ligases Endowed with Unnatural Substrate Specificity for Targeted Protein Silencing,” J Bio Chem. Vol 289, No. 11, pp. 7844-7855, Mar. 14, 2014, which is incorporated herein by reference in its entirety.

As used herein, the term “ubiquitin ligase”, also known as E3 ligase, refers to a family of proteins that facilitate the transfer of ubiquitin, alone or in complex, to a specific substrate protein, therefore targeting the substrate protein for degradation. E3 ubiquitin ligases fall into five classes, two major classes (HECT (homologous to E6-AP carboxyl terminus) catalytic domain E3 ligases and zinc-binding RING (really interesting new gene) finger adaptor domain E3 ligases) and three minor classes (U Box, PHD, and HUL-1 E3 ligases. For example, neural precursor cell expressed developmentally down-regulated protein 4 (NEDD4) is a HECT domain E3 ligase multiple roles in cellular signalling. Ubiquitination by NEDD4 involves initial binding of a ubiquitin-conjugated E2 to NEDD4's HECT domain with subsequent thiol-ester exchange to transfer the ubiquitin moiety from the E2 to the catalytic cysteine in NEDD4. The conjugated ubiquitin moiety is then transferred to either a target protein or another ubiquitin molecule to promote a polyubiquitin chain. In both cases, the ubiquitin is transferred from the E3 catalytic cysteine to a lysine side-chain ε-amino group. Polyubiquitination can mark proteins for degradation by the proteasome.

As used herein, the term “ubiquitination” refers to the attachment of the protein ubiquitin to lysine residues of other molecules. Ubiquitination of a molecule, such as a peptide or protein, can act as a signal for its rapid cellular degradation, and for targeting to the proteasome complex.

As used herein, the term “variable” refers to the fact that certain segments of the variable domains differ extensively in sequence among antibodies. The variable domain mediates antigen binding and defines specificity of a particular antibody for its particular antigen. However, the variability is not evenly distributed across the amino acid span of the variable domains. Instead, the variable domain regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies. See Kabat et al, (1991). The constant domains are not involved directly in binding an antibody to an antigen, but exhibit various effector functions, such as participation of the antibody in antibody dependent cellular cytotoxicity (ADCC).

As used herein, the term “variants” include variants with similar amino acid or nucleotide sequences to the original (e.g. wild-type) sequences, but with one or more amino acid or nucleotide changes that result in a variant which still retains the function of the original protein from which they are derived. For example, a variant of the target protein described herein include variants that still facilitate sufficient binding to the compound which enables ubiquitination of the polypeptide sequence via the ubiquitin ligase.

### Ubiquitin-Protease Pathway (UPP)

Ubiquitin is a small (about 8.5 kDa) protein that has been found in most tissues of eukaryotic organisms. The addition of ubiquitin to a substrate protein is called ubiquitination or ubiquitylation. Ubiquitination can affect proteins in many ways, including signaling for their degradation via the proteasome. Ubiquitin is covalently coupled to a substrate lysine by activity of an E1 (ubiquitin activating enzyme), E2 (ubiquitin conjugating enzyme) and E3 (ubiquitin ligase) enzyme cascade. A E3 ubiquitin RING ligase is a protein that recruits an E2 ubiquitin-conjugating enzyme that has been loaded with ubiquitin, therefore E3 ubiquitin RING ligases interact with both the target protein and the E2 enzyme. The protein substrate is then ubiquitinated and subsequently degraded by proteasomes.

An E3 ubiquitin ligase, e.g., cereblon, can ubiquitinate a target protein once the target protein is placed in proximity to the E3 ubiquitin ligase. The ARRDC1 fusion proteins and the ARMMs disclosed herein are engineered to bring a ubiquitin ligase into proximity with a target protein, promoting ubiquitination of the target protein and ultimately its targeted degradation by the proteosome pathway. Thus, in some aspects, the present disclosure provides proximity induced ubiquitization and, subsequently, degradation. The disclosure provides multiple approaches to achieve the desired juxtaposition of target protein and ubiquitin ligase, including embodiments that associate a targeting domain with ARRDC1 covalently (e.g., a fusion with ARRDC1) or noncovalently (e.g., an aptamer that is bound to ARRDC1 via an RNA binding protein that is fused to ARRDC1) to bring the target protein in proximity to a ubiquitin ligase that is recruited by ARRDC1, as well as embodiments that supply the ubiquitin ligase activity, such as by fusion of a ubiquibody (e.g., a construct comprising a targeting domain and a ubiquitin ligase) to ARRDC1 or fusion of a targeting domain to a Fc region and an RNA binding protein that binds a target RNA that encodes a ubiquitin ligase (e.g. TRIM21).

### Ubiquitin Ligase

As mentioned previously, the E3 ubiquitin ligase can recruit the E2 ubiquitin-conjugating enzyme that is loaded with ubiquitin, recognize a protein substrate, and facilitate the transfer of ubiquitin from E2 to the target protein. The E3 ubiquitin ligase largely determines the substrate specificity. There are several hundred ubiquitin protein ligases in mammals. These can be classified into two main groups: the RING E3s which mediate the direct transfer of ubiquitin to the substrate. and the HECT E3s, including NEDD4 family members, which are involved in the transfer of activated ubiquitin from the E2 to the substrate by forming an intermediate complex with the C-terminus of the E3. See N. Boase and S Kumar, “NEDD4: The founding member of a family of ubiquitin-protein ligases,” Gene, 2015 Feb. 25; 557(2): 113-122. Exemplary ubiquitin ligases include, but are not limited to, HECW2 (HECT, C2 And WW Domain Containing E3 Ubiquitin Protein Ligase 2), HECW1 (HECT, C2 And WW Domain Containing E3 Ubiquitin Protein Ligase 1), WWPL (WW Domain Containing E3 Ubiquitin Protein Ligase 1), WWP2 (WW domain-containing Protein 2), NEDD4-1 (Neural Precursor Cell Expressed Developmentally Downregulated 4-1), NEDD4-2 (Neural Precursor Cell Expressed Developmentally Downregulated 4-2), NEDD4L (NEDD4 Like E3 Ubiquitin Protein Ligase), SMURFL (Smad Ubiquitination Regulatory Factor-L), SMURF2 (Smad Ubiquitin Regulatory Factor 2), SMURF1 (Smad Ubiquitin Regulatory Factor 1), ITCH, NEDL1 (HECT-Type Ubiquitin-Protein Isopeptide Ligase-1), NEDL2 (HECT-Type Ubiquitin-Protein Isopeptide Ligase-2), and TRIM21 (Tripartite Motif-Containing Protein 21).

### Ubiquibody

In some embodiments, the ARRDC1 protein or a variant thereof is fused to a ubiquitin ligase. In some embodiments, the ubiquitin ligase is a ubiquibody. Ubiquibodies are engineered protein chimeras that combine the ubiquitination activity of E3 ubiquitin ligases with the binding affinity and substrate specificity of designer binding proteins (DBPs). See Morgan Baltz, Erin Stephens, and Matthew DeLisa, “Design and Functional Chracterization of Synthetic E3 Ubiquitin Ligases for Targeted Protein Depletion: selective protein knockout using enginnered ubiquibodies,” Curr Protoc Chem Biol., 2018 March; 10(1): 72-90. The ubiqubodies facilitate the redirection and proteolytic degradation of specific substrate targets, which may not otherwise be bound for the proteasome. The ubiquibodies may comprise a degradation domain (e.g., a ubiquitin ligase catalytic domain) and a targeting domain capable of specifically directing the degradation domain to a substrate where the targeting domain is heterologous to the degradation domain. A linker can couple the degradation domain to the targeting domain. In some embodiments, the degradation domain comprises a eukaryotic U-box motif. Additional information regarding ubiquibodies can be found in WO2012135284, which is incorporated herein by reference, in its entirety.

### ARRDC1 Protein

ARRDC1 is a protein that comprises a PSAP (SEQ ID NO: 1) and a PPXY (SEQ ID NO: 2) motif, also referred to herein as a PSAP and PPXY domain, respectively, in its C-terminus. Exemplary, non-limiting ARRDC1 protein sequences are provided herein, and additional, suitable ARRDC1 protein sequences, isoforms, and fragments according to aspects of this invention are known in the art. It will be appreciated by those of skill in the art that this invention is not limited in this respect. Additional information regarding ARRDC1 is provided in U.S. Pat. No. 10,945,954 and U.S. Pat. No. 10,260,055, which are incorporated by reference herein. Exemplary ARRDC1 sequences include the following (PSAP and PPXY motifs are marked):

>gi|22748653|NP_689498.1|arrestin domain-containing protein 1
[Homo sapiens]
(SEQ ID NO: 3)
mgrvqlfesilshgrvvyspgeplagtvrvrlgaplpfrairvtcigscgvsnkandt
awvveegyfnsslsladkgslpagehsfpfqfllpataptsfegpfgkivhqvraaih
tprfskdhkcslvfyilsplnlnsipdieqpnvasatkkfsyklvktgsvvltastdlr
gyvvgqalqlhadvenqsgkdtspvvasllqkvsykakrwihdvrtiaevegagv
kawrraqwheqilvpalpqsalpgcslihidyylqvslkapeatvtlpvfigniavnh

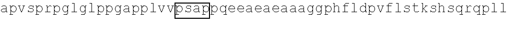

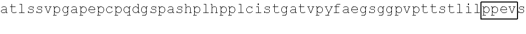

swgypyeappsyeqscggvepsltpes
>gi|244798004|ref|NP_001155957.1|arrestin domain-containing
protein 1 isoform a [Mus musculus]
(SEQ ID NO: 4)
mgrvqlfeirlsqgrvvygpgeplagtvhlrlgaplpfrairvtcmgscgvstkand
gawvveesyfnsslsladkgslpagehnfpfqfllpataptsfegpfgkivhqvrasi
dtprfskdhkcslvfyilsplnlnsipdieqpnvasttkkfsyklvktgnvvltastdl
rgyvvgqvlrlrqadienqsgkdtspvvasllqkvsykakrwiydvrtiaevegtgv
kawrraqwqeqilvpalpqsalpgcslihidyylqvsmkapeatvtlplfvgniavn

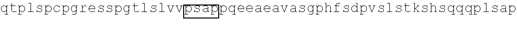

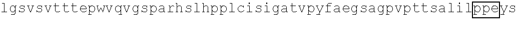

swgypyeappsyeqscgaagtdlglipgs
>gi|244798112|ref|NP_848495.2|arrestin domain-containing
protein 1 isoform b [Mus musculus]
(SEQ ID NO: 5)
mgrvqlfeirlsqgrvvygpgeplagtvhlrlgaplpfrairvtcmgscgvstkand
gawvveesyfnsslsladkgslpagehnfpfqfllpataptsfegpfgkivhqvrasi
dtprfskdhkcslvfyilsplnlnsipdieqpnvasttkkfsyklvktgnvvltastdl
rgyvvgqvlrlqadienqsgkdtspvvasllqvsykakrwiydvrtiaevegtgvk
awrraqwqeqilvpalpqsalpgcslihidyylqvsmkapeatvtlplfvgniavnq

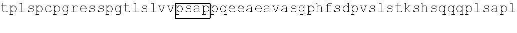

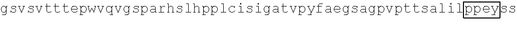

wgypyeappsyeqscgaagtdlglipgs

ARRDC1 PPXY motifs have been shown to recruit E3 ubiquitin ligases from the NEDD4 family, including but not limited to, HECW2, HECW1, NEDD4, NEDD4L, SMURF2, SMURF1, WWP1, WWP2, and ITCH. Without limited to any specific theory, proximity of a target protein to ubiquitin ligase bound to ARRDC1 may ubiquitinate the target protein and drive its degradation by the proteasome.

In some embodiments, the ARRDC1 protein or a variant is an ARRDC1 protein fragment. In some embodiments, the ARRDC1 protein fragment is a C-terminal ARRDC1 protein fragment. In some embodiments, the ARRDC1 protein fragment comprises the PSAP motif and at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 contiguous amino acids of the ARRCD1 sequence. Additional information can be found in U.S. Pat. No. 10,945,954, which is incorporated herein by reference in its entirety

In some embodiments, the ARRDC1 protein or a variant thereof comprises at least two PPXY motifs. In some embodiments, the ARRDC1 protein or a variant thereof comprises at least three PPXY motifs. In some embodiments, the ARRDC1 protein or a variant thereof comprises at least four PPXY motifs. In some embodiments, the ARRDC1 protein or a variant thereof comprises at least five PPXY motifs. In some embodiments, the ARRDC1 protein or a variant thereof comprises at least six PPXY motifs. In some embodiments, the ARRDC1 protein or a variant thereof comprises at least two but less than ten PPXY motifs. In some embodiments, the ARRDC1 protein or a variant thereof comprises at least two but less than six PPXY motifs. In some embodiments, the ARRDC1 protein or a variant thereof comprises at least two but less than four PPXY motifs. In some embodiments, the ARRDC1 protein or a variant thereof comprises two, three, four, five or six PPXY motifs. In some embodiments, the PPXY motifs are the same. In some embodiments, at least some of the PPXY motifs are different. In some embodiments, all the PPXY motifs are different from each other, that is, no PPXY motif is the same as another PPXY motif in the ARRDC1 protein or a variant thereof. In some embodiment, the ARRDC1 protein or a variant thereof comprises a PSAP motif.

### ARRDC1 Fusion Proteins

Provided herein are ARRDC1 fusion proteins comprising an ARRDC1 protein or a variant thereof, and a non-enzymatic targeting domain that recruits a target protein. In some embodiments. the targeting domain is fused to the N-terminal end of the ARRDC1 protein or a variant thereof. In some embodiments, the targeting domain is fused to the C-terminal end of the ARRDC1 protein or a variant thereof. In some embodiments, the ARRDC1 protein or a variant thereof is covalently bound to the targeting domain. In some embodiments, the ARRDC1 protein or a variant thereof is non-covalently bound to the targeting domain. In some embodiments, the ARRDC1 protein or a variant thereof is fused to the targeting domain by a linker.

In some embodiments, the non-enzymatic targeting domain comprises an interaction domain. In some embodiments, the interaction domain comprises a non-enzymatic protein binding peptide. In some embodiments, the non-enzymatic protein binding peptide comprises an antibody, an antibody derivative or an antibody fragment. In some embodiments, the non-enzymatic protein binding peptide comprises an intrabody. In some embodiments, the intrabody comprises a single chain variable region, a nanobody, or a single domain antibody. In some embodiments, the single domain antibody is a camelid single domain. In some embodiments, the non-enzymatic protein binding peptide comprises a ligand or a ligand fragment. In some embodiments, the non-enzymatic protein binding peptide comprises an antigen or an antigen fragment. In some embodiments, the non-enzymatic targeting domain comprises an aptamer. In some embodiments, the non-enzymatic targeting domain comprises an alternative scaffold protein, such as an engineered protein based on an ankyrin repeat motif (e.g., DARPin), an engineered protein based on the Z domain of Protein A (e.g., an affibody), an engineered protein based on a fibronectin type III domain protein (e.g., adnectin. trinectin, monobody or centyrin), an engineered protein based on a an SH3 domain, such as the SH3 domain of the protooncogene fyn (e.g., a fynomer), an engineered protein based on a lipocalin (e.g., an anticalin). In some embodiments, a terminal of the non-enzymatic targeting domain is fused to at least one additional PPXY motif. In some embodiments, a terminal of the non-enzymatic targeting domain is fused to at least two additional PPXY motifs. In some embodiments, a terminal of the non-enzymatic targeting domain is fused to at least three additional PPXY motifs. In some embodiments. a terminal of the non-enzymatic targeting domain is fused to at least four additional PPXY motifs. In some embodiments, a terminal of the non-enzymatic targeting domain is fused to at least one but less than ten additional PPXY motifs. In some embodiments, a terminal of the non-enzymatic targeting domain is fused to at least one but less than eight additional PPXY motifs. In some embodiments, a terminal of the non-enzymatic targeting domain is fused to at least one but less than six additional PPXY motifs. In some embodiments, a terminal of the non-enzymatic targeting domain is fused to at least two but less than six additional PPXY motifs. In some embodiments, a terminal of the non-enzymatic targeting domain is fused to at least two but less than four additional PPXY motifs. In some embodiments, a terminal of the non-enzymatic targeting domain is fused to one additional PPXY motif. In some embodiments, a terminal of the non-enzymatic targeting domain is fused to two additional PPXY motifs. In some embodiments, a terminal of the non-enzymatic targeting domain is fused to three additional PPXY motifs. In some embodiments, a terminal of the non-enzymatic targeting domain is fused to four additional PPXY motifs. In some embodiments, the additional PPXY motifs are the same. In some embodiments, at least some of the additional PPXY motifs are different. In some embodiments, all of the additional PPXY motifs are different, that is, no additional PPXY motif is the same as another additional PPXY motif fused to non-enzymatic targeting domain. In some embodiments, the terminal of the non-enzymatic targeting domain is the N-terminal. In some embodiments, the terminal fo the non-enzymatic targeting domain is the C-terminal.

In some embodiments, the target protein is an enzyme, a transcription factor, a tumor promoter or the protein product of an oncogene, a developmental regulator, a growth factor, a metastasis promoter, an anti-apoptotic protein, a membrane-associated protein, a transmembrane receptor, an enzyme, a nuclease, a recombinase, a reprogramming factor, or a membrane associated protein.

### RNA Binding Protein

In some embodiments, the non-enzymatic targeting domain is fused to a Fc region of an antibody. In some embodiments, the non-enzymatic targeting domain or the Fc region of an antibody is fused to an RNA binding protein.

In some embodiments, the RNA binding protein is a naturally-occurring protein, or non-naturally-occurring variant thereof, or a non-naturally occurring protein that binds to an RNA, for example, an RNA with a specific sequence or structure.

In certain embodiments, the RNA binding protein is a trans-activator of transcription (Tat) protein that specifically binds a trans-activating response element (TAR element). An exemplary Tat protein comprises the amino acid sequence as set forth in SEQ ID NO: 6 (Table 1). Exemplary amino acid sequences of Tat proteins, as well as Tat protein fragments that bind TAR elements, are shown in Table 1. In some embodiments, the RNA binding protein is at least 60%, 65%, 70%, 75%, 80%, 85%, 90%, 95%, 98%, or 99% identical to the amino acid sequence of any one of SEQ ID NOs: 6-25 and binds a TAR element. In some embodiments, the RNA binding protein has at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, or at least 130 identical contiguous amino acids of any one of SEQ ID NOs: 6-25, and binds a TAR element. In some embodiments, the RNA binding protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 6-25, and binds a TAR element. In some embodiments, the RNA binding protein comprises any one of the amino acid sequences set forth in SEQ ID NOs: 6-25. In some embodiments, the Tat protein comprises an amino acid sequence as set forth in any one of SEQ ID NOs: 6-25. The RNA binding protein may also be a variant of a Tat protein that is capable of associating with a TAR element. Tat proteins, as well as variants of Tat proteins that bind to a TAR element, are known in the art and have been described previously, for example, in Kamine et al., “Mapping of HIV-1 Tat Protein Sequences Required for Binding to Tar RNA”, Virology 182, 570-577 (1991); and Patel, “Adaptive recognition in RNA complexes with peptides and protein modules” Curr Opin Struct Biol. 1999 February; 9(1):74-87; the entire contents of each of which are incorporated herein by reference. In some embodiments, the Tat protein is an HIV-1 Tat protein, or variant thereof. In some embodiments, the Tat protein is bovine immunodeficiency virus (BIV) Tat protein, or variant thereof.

The Tat protein consists of several domains, one is a short lysine and arginine rich region important for nuclear localization. The nine amino acid basic region of HIV-1 Tat is found at positions 49-57 of SEQ ID NO: 6 and is capable of binding a TAR element. In some embodiments, the Tat sequence comprisess the nine amino acid basic region of Tat (SEQ ID NO: 14). In some embodiments the RNA binding protein comprises any one of the amino acid sequences as set forth in SEQ ID NOs: 6-8, 10, 11, or 14-25. In some embodiments, the Tat proteins are fusion proteins.

TABLE 1
Exemplary amino acid sequences of Tat proteins and fragments

| | | SEQ ID |
|---|---|---|
| Tat (Residue NOs) | Sequence | NO |
| HIV-1 Tat (1-101) | MEPVDPRLEPWKHPGSQPRT PCTTCYCKKC | 6 |
| | CFHCQVCFTT KALGISYGRK KRRQRRRPPQ | |
| | GSQTHQVSLS KQPSSQPRGD QTGPKESKKK | |
| | VERETEADPKP | |
| HIV-1 Tat (1-86) | MEPVDPRLEP WKHPGSQPRT PCTTCYCKKC | 7 |
| | CFHCQVCFTT KALGISYGRK KRRQRRRPPQ | |
| | GSQTHQVSLS KQPSSQPRGD QTGPKE | |
| HIV-1 Tat (37-72) | CFTT KALGISYGRK KRRQRRRPPQ GSQTHQVSLS | 8 |
| | KQ | |
| HIV-1 Tat (1-45) | MEPVDPRLEP WKHPGSQPRT PCTTCYCKKC | 9 |
| | CFHCQVCFTT KALGI | |
| HIV-1 Tat (49-86) | RK KRRQRRRPPQ GSQTHQVSLS KQPSSQPRGD | 10 |
| | QTGPKE | |
| HIV-1 Tat (52-86) | RRQRRRPPQ GSQTHQVSLS KQPSSQPRGD | 11 |
| | QTGPKE | |
| HIV-1 Tat (55-86) | RRRPPQ GSQTHQVSLS KQPSSQPRGD QTGPKE | 12 |
| HIV-1 Tat (58-86) | PPQ GSQTHQVSLS KQPSSQPRGD QTGPKE | 13 |
| HIV-1 Tat (49-57) | RK KRRQRRR | 14 |
| HIV-1 Tat (49-59) | RK KRRQRRRPP | 15 |
| HIV-1 Tat (49-61) | RK KRRQRRRPPQ G | 16 |
| HIV-1 Tat (49-63) | RK KRRQRRRPPQ GSQ | 17 |
| HIV-1 Tat (49-65) | RK KRRQRRRPPQ GSQTH | 18 |
| HIV-1 Tat (37-57) | CFTT KALGISYGRK KRRQRRR | 19 |
| HIV-1 Tat (38-62) | CFTT KALGISYGRK KRRQRRRPPQ GSQ | 20 |
| HIV-1 Tat (47-58) | GRRK KRRQRRRP | 21 |
| HIV-1 Tat (46-65) | RK KRRQRRRPPQ GSQTH | 22 |
| HIV-2 Tat (1-130) | METPLKAPEG SLGSYNEPSS CTSEQDAAAQ | 23 |
| | GLVSPGDEIL YQLYQPLEAC DNKCYCKKCC | |
| | YHCQMCFLNK GLGIWYERKG RRRRTPKKTK | |
| | AHSSSASDKS ISTRTGNSQP EKKQKKTLET | |
| | ALETIGGPGR | |
| BIV Tat | MPGPWVAMIM LPQPKESFGG KPIGWLFWNT | 24 |
| | CKGPRRDCPH CCCPICSWHC QLCFLQKNLG | |
| | INYGSGPRRR GTRGKGRRIR RTASGGDQRR | |
| | EADSQRSFTN MDQ | |
| BIV Tat | SGPRPRGTRGKGRRIRR | 25 |

In some embodiments, the RNA binding protein is a regulator of virion expression (Rev) protein (e.g., Rev from HIV-1), or variant thereof, that hinds to a Rev response element (RRE). Rev proteins are known in the art and are known to the skilled artisan. For example, Rev proteins have been described in Fernandes et al., “The HIV-1 Rev response element: An RNA scaffold that directs the cooperative assembly of a homo-oligomeric ribonucleoprotein complex” RNA Biology 9:1, 6-11; January 2012; Cochrane et al., “The human immunodeficiency virus Rev protein is a nuclear phosphoprotein” Virology 171 (1):264-266, 1989; Grate et al., “Role REVersal: understanding how RRE RNA binds its peptide ligand” Structure. 1997 Jan. 15; 5(1):7-11; and Patel, “Adaptive recognition in RNA complexes with peptides and protein modules” Curr Opin Struct Biol. 1999 February; 9(1):74-87; the entire contents of each of which are incorporated herein by reference in their entirety. An exemplary Rev protein comprises the amino acid sequence as set forth in SEQ ID NOs: 28-30 (Table 2). In some embodiments, the RNA binding protein is at least 60%, 65%, 70%, 75%, 80%, 85%, 90%, 95%, 98%, or 99% identical to the amino acid sequence of any one of SEQ ID NOs: 28-30, and binds a Rev response element. In some embodiments, the RNA binding protein has at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or at least 115 identical contiguous amino acids of any one of SEQ ID NOs: 28-30, and binds a Rev response element. In some embodiments, the RNA binding protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 28-30, and binds a Rev response element. In some embodiments, the RNA binding protein comprises any one of the amino acid sequences set forth in SEQ ID NOs: 28-30. In some embodiments, the RNA binding protein comprises a variant of any one of the amino acid sequences as set forth in SEQ ID NOs: 28-30 that are capable of binding an RRE. Such variants would be apprarent to the skilled artisan based on this disclosure and knowledge in the art and may be tested (e.g. for binding to an RRE) using routine methods known in the art.

TABLE 2
Exemplary sequences of RRE/Rev proteins

| | | SEQ |
|---|---|---|
| | Sequence | ID NO |
| HIV-1 RRE | ggucugggcgcagcgcaagcugacgguacaggcc | 26 |
| HIV-1 RRE aptamer | ggcuggacucguacuucgguacuggagaaacagcc | 27 |
| HIV-1 Rev | MAGRSGDSDEELIRTVRLIKLLYQSNPPPNPEGTRQ | 28 |
| | ARRNRRRRWRERQRQIHSISERILGTYLGRSAEPVP | |
| | LQLPPLERLTLDCNEDCGTSGTQGVGSPQILVESPT | |
| | VLESGTKE | |
| HIV-1 Rev peptide | TRQARRNRRRRWRERQR | 29 |
| Evolved HIV-1 | RDRRRRGSRPSGAERRRRRAAAA | 30 |
| RRE-binding | | |
| peptide | | |

In some embodiments, the RNA binding protein is a coat protein of an MS2 bacteriophage that specifically binds to an MS2 RNA. MS2 phage coat proteins that specifically bind MS2 RNAs are known in the art. An exemplary MS2 phage coat protein comprises the amino acid sequence as set forth in SEQ ID NO: 34 (Table 3). In some embodiments, the RNA binding protein is at least 60%, 65%, 70%, 75%, 80%, 85%, 90%, 95%, 98%, or 99% identical to the amino acid sequence of SEQ ID NO: 34, and binds an MS2 RNA. In some embodiments, the RNA binding protein has at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or at least 115 identical contiguous amino acids of SEQ ID NO: 34, and binds an MS2 RNA. In some embodiments, the RNA binding protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to SEQ ID NO: 34, and binds an MS2 RNA. In some embodiments, the RNA binding protein comprises the amino acid sequence set forth in SEQ ID NO: 34. In some embodiments, the RNA binding protein comprises a fragment or variant of SEQ ID NO: 34 that is capable of binding to an MS2 RNA. Methods for testing whether variants or fragments of MS2 phage coat proteins bind to MS2 RNAs (e.g., SEQ ID NO: 34) can be performed using routine experimentation and would be apparent to the skilled artisan.

TABLE 3
MS2 sequences

| | | SEQ ID |
|---|---|---|
| MS2 | Sequence | NO |
| Bacteriophage MS2 RNA | acaugaggauuacccaugu | 31 |
| MS2 RNA | ccggaggaucaccacggg | 32 |
| MS2 RNA | ccacagucacuggg | 33 |
| Bacteriophage MS2 Coat | ASNFTQFVLVDNGGTGDVTVAPSNFANGVAEWIS | 34 |
| Protein | SNSRSQAYKVTCSVRQSSAQNRKYTIKVEVPKVAT | |
| | QTVGGVELPVAAWRSYLNMELTIPIFATNS DCELI | |
| | VKAMQ GLLKDGNPIP SAIAANSGIY | |

In some embodiments, the RNA binding protein is a P22 N protein (e.g., P22 N from bacteriophage), or variant thereof, that binds to a P22 boxB RNA. P22 N proteins are known in the art and would be apparent to the skilled artisan. An exemplary P22 N that specifically binds to a protein P22 boxB RNA comprises the amino acid sequence NAKTRRHERRRKLAIERDTI (SEQ ID NO: 35).

In some embodiments, the RNA binding protein is a λ N protein (e.g., λ N from bacteriophage), or variant thereof, that binds to a λ boxB RNA. λ N proteins are known in the art and would be apparent to the skilled artisan. An exemplary λ N protein that specifically binds to a X boxB comprises the amino acid sequence GSMDAQTRRRERRAEKQAQWKAAN (SEQ ID NO: 36).

In some embodiments, the RNA binding protein is a φ21 N protein (e.g., φ21 N from bacteriophage), or variant thereof, that binds to a φ21 boxB RNA. 21 N proteins are known in the art and would be apparent to the skilled artisan. An exemplary φ21 N protein that specifically binds to a φ21 boxB RNA comprises amino acid sequence GTAKSRYKARRAELIAERR (SEQ ID NO: 37). The N peptide binds as an a-helix and interacts predominately with the major groove side of the 5′ half of the boxB RNA stem-loop. This binding interface is defined by surface complementarity of polar and nonpolar interactions. The N peptide complexed with the exposed face of the (φ21 boxB loop is similar to the GNRA tetraloop-like folds of the related λ and P22 bacteriophage N peptide—boxB RNA complexes.

In some embodiments, the RNA binding protein is a HIV-1 nucleocapsid (e.g., nucleocapsid from HIV-1), or variant thereof, that hinds to a SL3 yr RNA. HIV-I nucleocapsid proteins are known in the art and would be apparent to the skilled artisan. An exemplary HIV-1 nucleocapsid that specifically binds to a SL3 xv RNA comprises amino acid sequence MQKGNFRNQRKTVKCFNCGKEGHIAKNCRAPRKKGCWKCGKEGHQMKDCTERQAN (SEQ ID NO: 38).

Additional information regarding the RNA binding proteins can be found in WO201867546, which is incorporated herein by reference in its entirety.

### Binding RNAs

Some aspects of the disclosure relate to RNA molecules that bind proteins. In some embodiments, the binding RNA is a naturally occurring RNA, or non-naturally occurring variant thereof, or a non-naturally occurring RNA, that binds to a protein having a specific amino acid sequence or structure.

In certain embodiments, the binding RNA is a trans-activating response element (TAR element), which is an RNA stem-loop structure that is found at the 5′ ends of nascent human immunodeficiency virus-1 (HIV-1) transcripts and specifically bind to a trans-activator of transcription (Tat) protein. In some embodiments, the TAR element is a bovine immunodeficiency virus (BIV) TAR. An exemplary TAR element comprises the nucleic acid sequence as set forth in SEQ ID NO: 25. Further exemplary TAR sequences can be found in Table 1; however, these sequences are not meant to be limiting and additional TAR element sequences that bind to a Tat protein, or variant thereof, are also within the scope of this disclosure. The binding RNA may also be a variant of a TAR element that is capable of associating with the RNA binding protein, trans-activator of transcription (Tat protein), which is a regulatory protein that is involved in transcription of the viral genome. Variants of TAR elements that are capable of associating with Tat proteins would be apparent to the skilled artisan based on this disclosure and knowledge in the art, and are within the scope of this disclosure. Further, the association between a TAR variant and a Tat protein, or Tat protein variant, may be tested using routine methods. TAR elements and variants of TAR elements that bind to Tat proteins are known in the art. In some embodiments, the binding RNA comprises the nucleic acid sequence as set forth in SEQ ID NOs: 39-44. In some embodiments, the binding RNA comprises a variant of any of the nucleic acid sequences set forth in SEQ ID NOs: 39-44 that are capable of binding to a Tat protein or variant thereof.

TABLE 4
TAR Sequences

| | | SEQ ID |
|---|---|---|
| TAR | Sequence | NO |
| HIV-1 TAR | gggucucucugguuagaccagaucugagccugggagcucucuggcuaa | 39 |
| RNA + | cuagggaacccacug | |
| 1-59 | | |
| ΔTAR | gggucucucugguuagaccagaucugagccugggcucuggcuaacuag | 40 |
| | ggaacccacug | |
| HIV-1TAR | gggucucucugguuagaccagaucugagccugggagcucucuggcuaa | 41 |
| | cuagggaacc | |
| HIV-1 TAR | agaucugagccugggagcucucu | 42 |
| Hybrid TAR | gcucguugagcucugggaagcuccgagc | 43 |
| BIV TAR | ucguguagcucauuagcuccga | 44 |

In some embodiments, the binding RNA is a Rev response element (RRE), or variant thereof, that binds to a Rev protein (e.g., Rev from HIV-1). Rev response elements are known in the art and would be apparent to the skilled artisan for use in the present invention. Exemplary RRE nucleic acid sequences that bind Rev include, without limitation, those nucleic acid sequences set forth in SEQ ID NOs: 26 and 27 (Table 2).

In some embodiments, the binding RNA is an MS2 RNA that specifically binds to a MS2 phage coat protein. Typically, the coat protein of the RNA bacteriophage MS2 binds a specific stem-loop structure in viral RNA (e.g., MS2 RNA) to accomplish encapsidation of the genome and translational repression of replicase synthesis. RNAs that specifically bind MS2 phage coat proteins are known in the art and would be apparent the skilled artisan. In some embodiments, an exemplary MS2 RNA that specifically binds to a MS2 phage coat protein comprises a nucleic acid sequence as set forth in any one of SEQ ID NOs: 31-33 (Table 3). In some embodiments, the binding RNA comprises the nucleic acid sequenc of any one of SEQ ID NOs: 31, 32, and 33.

In some embodiments, the binding RNA is an RNA that specifically hinds to a P22 N protein (e.g., P22 N from bacteriophage), or variant thereof. P22 N proteins are known in the art and would be apparent to the skilled artisan. An exemplary P22 boxB RNA that specifically binds to a P22 N protein comprises a nucleic acid sequence as set forth in gegcugacaaagcgc (SEQ ID NO: 45).

In some embodiments, the binding RNA is an RNA that specifically binds to a λ N protein (e.g., λ N from bacteriophage), or variant thereof. λ N proteins are known in the art and would be apparent to the skilled artisan. An exemplary A boxB RNA that specifically binds to a λ N protein comprises a nucleic acid sequence as set forth in gggcccugaagaagggccc (SEQ ID NO: 46).

In some embodiments, the binding RNA is an RNA that specifically binds to a (φ21 N protein (e.g., (φ21 N from bacteriophage), or variant thereof, φ21 N proteins are known in the art and would be apparent to the skilled artisan. An exemplary φ21 boxB RNA that specifically binds to a φ21 N protein comprises a nucleic acid sequence as set forth in ucucaaccuaaccguugaga (SEQ ID NO: 47).

In some embodiments, the binding RNA is an RNA that specifically binds to an HIV-1 nucleocapsid protein (e.g., nucleocapsid from HIV-1) or variant thereof. HIV-1 nucleocapsid proteins are known in the art and would be apparent to the skilled artisan. An exemplary RNA that specifically binds to a HIV-1 nucleocapsid comprises a nucleic acid sequence as set forth in ggacuagcggaggcuagucc (SEQ ID NO: 48).

It should be appreciated that the binding RNAs of the present disclosure need not be limited to naturally-occurring RNAs or non-naturally-occurring variants thereof, that have recognized protein binding partners. In some embodiments, the binding RNA may also be a synthetically produced RNA, for example an RNA that is designed to specifically bind to a protein (e.g., an RNA binding protein). In some embodiments, the binding RNA is designed to specifically bind to any protein of interest. In some embodiments, the binding RNA is an RNA produced by the systematic evolution of ligands by exponential enrichment (SELEX). SELEX methodology would be apparent to the skilled artisan and has been described previously, for example in U.S. Pat. Nos. 5,270,163; 5,817,785; 5,595,887; 5,496,938; 5,475,096; 5,861,254; 5,958,691; 5,962,219; 6,013,443; 6,030,776; 6,083,696; 6,110,900; 6,127,119; and 6,147,204; U.S. Appin 20030175703 and 20030083294. Potti et al., Expert Opin. Biol. Ther. 4:1641-1647 (2004), and Nimjec et al., Annu. Rev. Med. 56:555-83 (2005). The technique of SELEX has been used to evolve aptamers to have extremely high binding affinity to a variety of target proteins. See, for example. Trujillo U. H., et al., “DNA and RNA aptamers: from tools for basic research towards therapeutic applications”. Comb Chem High Throughput Screen 9 (8): 619-32 (2006) for its disclosure of using SELEX to design aptamers that bind vascular endothelial growth factor (VEGF). In some embodiments, the binding RNA is an aptamer that specifically binds a target protein.

Additional information regarding the binding RNAs can be found in WO201867546, which is incorporated herein by reference in its entirety.

### Linkers

In some embodiments, the ARRDC1 protein or a variant thereof is fused to the targeting domain via a linker. In some embodiments, the linker is a linker peptide. In some embodiments, the linker is cleavable.

In some embodiments, the linker peptide consists of about 100 amino acids. In some embodiments, the linker peptide consists of about 90 amino acids. In some embodiments, the linker peptide consists of about 80 amino acids. In some embodiments, the linker peptide consists of about 70 amino acids. In some embodiments, the linker peptide consists of about 60 amino acids. In some embodiments, the linker peptide consists of about 50 amino acids. In some embodiments, the linker peptide consists of about 40 amino acids. In some embodiments, the linker peptide consists of about 30 amino acids. In some embodiments, the linker peptide consists of about 20 amino acids. In some embodiments, the linker peptide consists of about 18 amino acids. In some embodiments, the linker peptide consists of about 15 amino acids. In some embodiments, the linker peptide consists of about 12 amino acids. In some embodiments, the linker peptide consists of about 11 amino acids. In some embodiments, the linker peptide consists of about 10 amino acids. In some embodiments, the linker peptide consists of about 9 amino acids. In some embodiments, the linker peptide consists of about 8 amino acids. In some embodiments, the linker peptide consists of about 7 amino acids. In some embodiments, the linker peptide consists of about 6 amino acids. In some embodiments, the linker peptide consists of about 5 amino acids. In some embodiments, the linker peptide consists of about 4 amino acids. In some embodiments, the linker peptide consists of about 3 amino acids. In some embodiments, the linker peptide consists of about 2 amino acids.

In some embodiments, the linker is from about I to about 10 amino acids in length. In some embodiments, the linker can be about 1 to about 5 amino acids in length. For example, the linker can contain 1, 2, 3, 4, or 5 amino acids. In some embodiments, the last amino acid of the linker contacting the C-terminus of the ARRDC1 protein or a variant and the N-terminal domain of a targeting domain is selected from the group consisting of Met, Cys, Thr, Arg, Lys, Ser, Gin, His, Ala, Tyr, Phe, Asn, Trp, Val, Leu, Asp, He, Gly, Glu and Pro.

In some embodiments, the linker peptide can be any linker peptide known by the skilled in the art. In some embodiments, the linker peptide has an amino acid sequence of GGGGS (SEQ ID NO: 49). In some embodiments, the linker peptide has an amino acid sequence of (GGGGS)₂ (SEQ ID NO: 50). In some embodiments, the linker peptide has an amino acid sequence of (GGGGS)₃ (SEQ ID NO: 51). In some embodiments, the linker peptide has an amino acid sequence of (GGGGS)₄ (SEQ ID NO: 52). In some embodiments, the linker peptide has an amino acid sequence of (Gly)₈ (SEQ ID NO: 53). In some embodiments, the linker peptide has an amino acid sequence of (Gly)₆ (SEQ ID NO: 54). In some embodiments, the linker peptide has an amino acid sequence of EAAAK (SEQ ID NO: 55). In some embodiments, the linker peptide has an amino acid sequence of (EAAAK)₂ (SEQ ID NO: 56). In some embodiments, the linker peptide has an amino acid sequence of (EAAAK)₃ (SEQ ID NO: 57). In some embodiments, the linker peptide has an amino acid sequence of A(EAAAK)₄ALEA(EAAAK)₄A (SEQ ID NO: 58). In some embodiments, the linker peptide has an amino acid sequence of PAPAP (SEQ ID NO: 59). In some embodiments, the linker peptide has an amino acid sequence of AEAAAKEAAAKA (SEQ ID NO: 60). In some embodiments, the linker peptide has an amino acid sequence of (Ala-Pro)_{n,} where n is 5 (SEQ ID NO: 61), 6 (SEQ ID NO: 91), 7 (SEQ ID NO: 92), 8 (SEQ ID NO: 93), 9 (SEQ ID NO: 94), 10 (SEQ ID NO: 95), 11 (SEQ ID NO: 96), 12 (SEQ ID NO: 97), 13 (SEQ ID NO: 98), 14 (SEQ ID NO: 99), 15 (SEQ ID NO: 100), 16 (SEQ ID NO: 101), or 17 (SEQ ID NO: 102). In some embodiments, the linker peptide is a disulfide. In some embodiments, the linker peptide has an amino acid sequence of VSQTSKLTRAETVFPDV (SEQ ID NO: 62). In some embodiments, the linker peptide has an amino acid sequence of PLGLWA (SEQ ID NO: 63). In some embodiments, the linker peptide has an amino acid sequence of RVLAEA (SEQ ID NO: 64). In some embodiments, the linker peptide has an amino acid sequence of EDVVCCSMSY (SEQ ID NO: 65). In some embodiments, the linker peptide has an amino acid sequence of GGIEGRGS (SEQ ID NO: 66). In some embodiments, the linker peptide has an amino acid sequence of TRHRQPRGWE (SEQ ID NO: 67). In some embodiments, the linker peptide has an amino acid sequence of AGNRVRRSVG (SEQ ID NO: 68). In some embodiments, the linker peptide has an amino acid sequence of RRRRRRRRR (SEQ ID NO: 69). In some embodiments, the linker peptide has an amino acid sequence of GFLG (SEQ ID NO: 70). In some embodiments, the linker peptide has an amino acid sequence of LE (SEQ ID NO: 71). In some embodiments, the linker peptide has an amino acid sequence of KESGSVSSEQLAQFRSLD (SEQ ID NO: 72). In some embodiments, the linker peptide has an amino acid sequence of EGKSSGSGSESKST (SEQ ID NO: 73). In some embodiments, the linker peptide has an amino acid sequence of GSAGSAAGSGEF (SEQ ID NO: 74). In some embodiments, the linker peptide has an amino acid sequence of (XP) n (SEQ ID NO: 75), where n is an integer greater than 0. In some embodiments, the linker peptide has an amino acid sequence of LEAGCKNFFPRSFTSCGSLE (SEQ ID NO: 76). In some embodiments, the linker peptide has an amino acid sequence of CRRRRRREAEAC (SEQ ID NO: 77).

In some embodiments, the linker peptide mimics the sequence of a peptide substrate, which is the target for a protease, such that the linker peptide can be well recognized by the protease active site. In some embodiments, the linker is non-immunogenic. In some embodiments, the linker peptide comprises an alpha-helical structure.

In some embodiments, the linker peptide comprises a protease cleavage site. In some embodiments, the protease cleavage site is a furin site or a variant thereof. In some embodiments, the linker peptide has an amino acid sequence of RXRR (SEQ ID NO: 78) or: RXKR (SEQ ID NO: 103). In some embodiments, the linker peptide has an amino acid sequence of TRHRQPRGWEQL (SEQ ID NO: 79). In some embodiments, the linker peptide has an amino acid sequence of RRRRRRRRR (SEQ ID NO: 80). In some embodiments, the linker peptide has an amino acid sequence of TRHRQPRGWE (SEQ ID NO: 81). In some embodiments, the linker peptide has an amino acid sequence of AGNRVRRSVG (SEQ ID NO: 82). In some embodiments the linker peptide has an amino acid sequence of RHRQPRGWEQL (SEQ ID NO: 83). In some embodiments, the protease cleavage site comprises a furin cleavage site.

In some embodiments comprising a linker peptide that includes a protease cleavage site, the protease cleavage site is a human immunodeficiency virus-1 (HIV-1) protease cleavage site. In some embodiments, the linker peptide comprises an amino acid sequence of SQNYIVQ (SEQ ID NO: 84) or SQNPIVQ (SEQ ID NO: 104). In some embodiments, the linker peptide has an amino acid sequence of NFSQNYIVQTG (SEQ ID NO: 85) or NFSQNPIVQTG (SEQ ID NO: 105). In some embodiments comprising a linker peptide that includes a protease cleavage site, the protease cleavage site is tobacco etch virus (TEV) protease cleavage site. In some embodiments, the linker peptide comprises an amino acid sequence of ENLYFQ (SEQ ID NO: 86) or ENLYFG (SEQ ID NO: 106). In some embodiments, the linker peptide comprsises an amino acid sequence of ENLYFQ (SEQ ID NO: 87) or ENLYFS (SEQ ID NO: 107). In some embodiments, the linker peptide comprises an amino acid sequence of GGGGSGGGGSGGGGSENLYFQGGGGSGGGGSGGGGS (SEQ ID NO: 88) or GGGGSGGGGSGGGGSENLYFGGGGGSGGGGSGGGGS (SEQ ID NO: 108). In some embodiments, the linker peptide comprises an amino acid sequence of GGGGSGGGGSGGGGSENLYFQGGGGSGGGGSGGGGS (SEQ ID NO: 89) or GGGGSGGGGSGGGGSENLYFSGGGGSGGGGSGGGGS (SEQ ID NO: 109).

### ARMMs

In some aspects, the present disclosure is related to a microvesicle that contains the ARRDC1 fusion protein as disclosed herein. An arrestin domain-containing protein 1 (ARRDC1)-mediated microvesicle (ARMM) disclosed herein comprises a lipid bilayer and an ARRDC1 fusion protein or a variant thereof. In some embodiments, such ARMMs are isolated from a subject, a biological sample, or a cell culture. In some embodiments, the ARMMs are prepared synthetically. Methods for generating and/or isolating ARMMs can be found in U.S. Pat. No. 9,737,480 and U.S. Pat. No. 9,816,080, which are incorporated by reference herein by their entirety. In some embodiments, the ARMM further comprises a fusion RNA, wherein the fusion RNA comprises an RNA sequence that binds the RNA binding protein of the ARRDC1 fusion protein and an expression sequence encoding a ubiquitin ligase or an aptamer. The RNA sequence can be any of the binding RNAs as disclosed herein. In some embodiments, the RNA sequence is a TAR element. In some embodiments, the TAR element is a bovine immunodeficiency virus (BIV) TAR. In some embodiments, the RNA sequence is a variant of the TAR element. In some embodiments, the RNA sequence is a Rev response element (RRE), or variant thereof. In some embodiments, the RNA sequence is an MS2 RNA. In some embodiments. the RNA sequence is an RNA that specifically hinds to a P22 N protein (e.g. P22 N from bacteriophage), or variant thereof. In some embodiments, the RNA sequence is an RNA that specifically binds to a λ N protein (e.g., λ N from bacteriophage), or variant thereof. In some embodiments, the RNA sequence is an RNA that specifically binds to a (φ21 N protein (e.g., (φ21 N from bacteriophage), or variant thereof. In some embodiments, the RNA sequence is an RNA that specifically binds to an HIV-1 nucleocapsid protein (e.g., nucleocapsid from HIV-1) or variant thereof. In some embodiments, the RNA sequence is a naturally-occuring RNA. In some embodiments, the RNA sequence is a non-naturally-occuring RNA. In some embodiments, the RNA sequence is a synthetically produced RNA.

In some embodiments, the ubiquitin ligase is Trim21, a variant or a fragment thereof. The expression sequence of Trim21 such as Trim21 mRNA is fused to the RNA sequence that binds the RNA binding protein of the ARRDC1 fusion protein. In some embodiments, the RNA sequence is fused to the expression sequence directly. In some embodiments, the RNA sequence is fused to the expression sequence by a linker.

In some embodiments, the ARMM further comprises a TSG101 protein or fragment thereof. In some embodiments, the TSG101 protein fragment comprises a TSG101 UEV domain. In some embodiments, the ARMM further comprises a cell surface protein (e.g., receptor) or a cytosolic protein (e.g., enzyme). In some embodiments, the ARMM further comprises an integrin, a receptor tyrosine kinase, a G-protein coupled receptor, or a membrane bound immunoglobulin. In some embodiments, the microvesicle comprises an integrin chosen from the group consisting of α1β1, α2β1, α4β1, α5β1, α6β1, αLβ2, αMβ2, αIIbβ3, αVβ3, αVβ5, αVβ6, and α6β4 integrins; a receptor tyrosine kinase chosen from the group consisting of an EGF receptor (ErbB family), insulin receptor, PDGF receptor, FGF receptor, VEGF receptor, HGF receptor, Trk receptor, Eph receptor, AXL 40 receptor, LTK receptor, TIE receptor, ROR receptor, DDR receptor, RET receptor, KLG receptor, RYK receptor, and MuSK receptor; a G-protein coupled receptor chosen from the group consisting of a rhodopsin-like receptor, secretin receptor, metabotropic glutamate/pheromone receptor, cyclic AMP receptor, frizzled/smoothened receptor, CXCR4, CCRS, or beta-adrenergic receptor; and/or an exocyst protein listed in Table 1 chosen from EXOC7, EXOC8, EXOC1, and EXOC2. Additional proteins can be found in U.S. Pat. No. 9,737,480, which is incorporated herein by reference in its entirety.

In some embodiments, the ARMM does not include an exosomal biomarker. In some embodiments, the ARMM does not include one or more exosomal biomarkers. In some embodiments, the exosomal biomarker is chosen from the group consisting of CD63, Lamp-i, Lamp-2, CD9, HSPA8, GAPDH, CD81, SDCBP, PDCD6IP, ENO1, ANXA2, ACTB, YWHAZ, H5P90AA1, ANXAS, EEF1A1, YWHAE, PPIA, MSN, CFL1, ALDOA, PGK1, EEF2, ANXA1, PKM2, HLA-DRA, and YWHAB.

In some embodiments, the ARMMs comprises an agent, for example, a protein, a nucleic acid, or a small molecule. In some embodiments, the agent is conjugated to the ARRDC1 protein or the ARRDC1 variant. In some embodiments, the nucleic acid is an RNA. In some embodiments, the nucleic acid is an RNAi agent. In some embodiments, the nucleic acid is a coding RNA, a non-coding RNA, an antisense RNA, an mRNA, a small RNA, an siRNA, an shRNA, a microRNA, an snRNA, a snoRNA, a lincRNA, a structural RNA, a ribozyme, or a precursor thereof. In some embodiments, the nucleic acid is a DNA. In some embodiments, the nucleic acid comprises a restrotransposon sequence, a LINE sequence, a SINE sequence, a composite SINE sequence, or an LTR-retrotransposon sequence. In some embodiments, the nucleic acid encodes a protein. In some embodiments, the agent comprises a detectable label. In some embodiments, the agent is a therapeutic agent. In some embodiments, the agent is a drug approved for human or veterinary use by a governmental agency. In some embodiments, the agent is a cytotoxic agent. In some embodiments, the agent is a protein. In some embodiments, the agent is a transcription factor, a transcriptional repressor, a fluorescent protein, a kinase, a phosphatase, a protease, a ligase, or a recombinase. In some embodiments, the agent is a small molecule. In some embodiments, the agent is covalently bound to the ARRDC1 protein or a variant thereof, or another protein of the ARMM. In some embodiments, agent is conjugated to the ARRDC1 protein or a variant thereof, or other protein via a linker. In some embodiments, the linker is a cleavable linker. In some embodiments, the linker comprises a protease recognition site. In some embodiments, the linker is a UV-cleavable linker. In certain embodiments, the linker is cleaved under specific conditions such as pH, redox conditions, etc. In some embodiments, the microvesicle diameter is from about 30 nm to about 500 nm.

### Nucleic Acid Sequences and Expression Construct

Some aspects of the disclosure provide nucleic acid sequences that encode any of the fusion protein described herein. For example, the nucleic acid sequence encodes an ARRDC1 fusion protein or a variant thereof fused to a non-enzymatic targeting domain that recruits a target protein. The targeting domain may be fused to the N-terminal end of the ARRDC1 protein or a variant thereof. The targeting domain may be fused to the C-terminal end of the ARRDC1 protein or a variant thereof. In some embodiments, the ARRDC1 protein or a variant is fused to the targeting domain by a linker as disclosed herein. The linker may be a cleavable linker. For example, the linker may comprise a protease cleavage site, such as a furin cleavage site.

In some aspects, an expression construct is provided herein, comprising a nucleotide sequence encoding an ARRDC1 fusion protein disclosed herein operably linked to a heterologous promoter. Such expression constructs are useful for generating the ARRDC1 fusion proteins disclosed herein in a cell, which, in turn, induces or increases the ARMM production in the cell. An ARMM produced by a cell expressing such an ARRDC1 fusion protein will, in some embodiments, comprise the ARRDC1 fusion protein encoded by the expression construct.

Some aspects of the disclosure further provide additional nucleic acid sequences that encode additional molecules intended for expression in parallel with the fusion proteins disclosed herein. Such additional molecules include RNA polymers comprising an RNA sequence that binds said RNA binding protein and an expression sequence encoding (i) a ubiquitin ligase, such as Trim21, or (b) an aptamer that specifically binds to a target protein.

### Microvesicle-Producing Cells

In some aspect, the present disclosure provides a microvesicle-producing cell that comprises a recombinant expression construct encoding the ARRDC1 fusion protein disclosed hrerein, under the control of a heterologous promoter. In some embodiments, the microvesicle-producing cell further comprises a recombinant expression construct encoding a fusion RNA disclosed herein under the control of a heterologous promoter, wherein the fusion RNA comprises (i) an RNA sequence that binds said RNA binding protein and (ii) an expression sequence encoding (a ubiquitin ligase or (b) an aptamer that specifically binds a target protein. In some embodiments, the RNA sequence is a TAR element. In some embodiments, the TAR element is a bovine immunodeficiency virus (BIV) TAR. In some embodiments, the RNA sequence is a variant of the TAR element. In some embodiments, the RNA sequence is a Rev response element (RRE), or variant thereof. In some embodiments, the RNA sequence is an MS2 RNA. In some embodiments, the RNA sequence is an RNA that specifically hinds to a P22 N protein (e.g., P22 N from bacteriophage), or variant thereof. In some embodiments, the RNA sequence is an RNA that specifically binds to a λ N protein (e.g., λ N from bacteriophage), or variant thereof. In some embodiments, the RNA sequence is an RNA that specifically binds to a (φ21 N protein (e.g., (φ21 N from bacteriophage), or variant thereof. In some embodiments, the RNA sequence is an RNA that specifically binds to an HIV-1 nucleocapsid protein (e.g., nucleocapsid from HIV-1) or variant thereof. In some embodiments, the RNA sequence is a naturally-occuring RNA. In some embodiments, the RNA sequence is a non-naturally-occuring RNA. In some embodiments, the RNA sequence is a synthetically produced RNA. In some embodiments, the ubiquitin ligase is Trim21. The expression sequence of Trim21 such as Trim21 mRNA is fused to the RNA sequence that binds the RNA binding protein of the ARRDC1 fusion protein. In some embodiments, the RNA sequence is fused to the expression sequence directly. In some embodiments, the RNA sequence is fused to the expression sequence by a linker.

In some embodiments, the expression of the ARRDC1 fusion protein induces or increases ARMM production of the cell. In some embodiments, the cell expresses or contains an agent in its cytoplasm or its plasma membrane that is included in ARMMs produced by the cell. In some embodiments, the cell is a eukaryotic cell. In some embodiments, the cell is a mammalian cell. In some embodiments, the cell is a human cell. In some embodiments, the cell further comprises a recombinant expression construct encoding an RNAi agent. In some embodiments, the RNAi agent is a nucleic acid. In some embodiments, the nucleic acid is a non-coding RNA, an antisense RNA, a small RNA, an siRNA, an shRNA, a microRNA, an snRNA, a snoRNA, a lincRNA, or a precursor thereof. In some embodiments, the cell further comprises a recombinant expression construct encoding a ribozyme.

In some embodiments, the cell has a reduced expression level of ubiquitin ligase as compared to a wildtype cell. In some embodiments, the cell is genetically engineered to have a reduced expression level of ubiquitin ligase as compared to a wildtype cell. In some embodiments, the expression of the ubiquitin ligase in the cell is reduced or inhibited by a small interference RNA (siRNA), CRISPR, or other gene editing tools known by those skilled in the art. In some embodiments, the expression of the ubiquitin ligase in the cell is reduced or inhibited by a small molecular inhibitor known by those skilled in the art. In some embodiments, the activity of the ubiquitin ligase in the cell is reduced or inhibited. In some embodiments, the activity of the ubiquitin ligase in the cell is reduced or inhibited by a small interference RNA (siRNA), CRISPR, or other gene editing tools known by those skilled in the art. In some embodiments, the activity of the ubiquitin ligase in the cell is reduced or inhibited by a small molecular inhibitor known by those skilled in the art. In some embodiments, the amount or activity of the ubiquitin ligase expressed in the cell is so minimal that it does not substantially interfer with the production of ARMMs in the cell. In some embodiments, the cell lacks the ubiquitin ligase. That is, the expression level of the ubiquitin ligase cannot be detected by tools available in the art. In some embodiments, the cell lacks at least one ubiquitin ligase. In some embodiments, the cell lacks one or more ubiquitin ligase. The ubiquitin ligase may be selected from the group consisting of HECW2, HECW1, WWPL, WWP2, NEDD4-1, NEDD4-2, NEDD4L, SMURFL, SMURF2, SMURF1, ITCH, NEDL1 and NEDL2.

In some embodiments, the cell has a reduced expression level of the target protein as compared to a wildtype cell. In some embodiments, the cell is genetically engineered to have a reduced expression level of the target protein as compared to a wildtype cell. In some embodiments, the expression of the target protein in the cell is reduced or inhibited by a small interference RNA (siRNA), CRISPR, or other gene editing tools known by those skilled in the art. In some embodiments, the expression of the the target protein in the cell is reduced or inhibited by a small molecular inhibitor known by those skilled in the art. In some embodiments, the amount of the target protein expressed in the cell is so minimal that it does not substantially interfer with the production of ARMMs in the host cell. In some embodiments, the cell lacks the target protein. That is, the expression level of the target protein cannot be detected by tools available in the art.

### Target Proteins

The ARRDC1 fusion protein or ARMMs disclosed herein may recruit a target protein for ubiquitination and degradation. Any protein, which can be recruited and/or bound to the targeting domain and acted on or degraded by a ubiquitin ligase can be a target protein. Generally, target proteins may include, for example, structural proteins, receptors, enzymes, cell surface proteins, proteins pertinent to the integrated function of a cell, including proteins involved in catalytic activity, aromatase activity, motor activity, helicase activity, metabolic processes (anabolism and catabolism antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, translation regulator activity. Proteins of interest can include proteins from curkaryotes and prokaryotes including humans as targets for drug therapy, other animals, including domesticated animals, microbials for the determination of targets for antibiotics and other antimicrobials and plants, and even viruses, among numerous others.

The target protein may be any protein that is desired to be degraded or reduced in a target cell. A target protein may be an enzyme, a transcription factor, a developmental regulator, a growth factor, a nuclease, a recombinase, a reprogramming factor, or a membrane associated protein, a tumor promoter or the protein product of an oncogene, a developmental regulator, a growth factor, a metastasis promoter, an anti-apoptotic protein, a membrane-associated protein, a transmembrane receptor, an enzyme or pathogenic protein (e.g., a pathogenic variant or mutation of a normal protein or a protein that has pathogenic or deleterious activity as a result of post-translational modification). The target protein may be an antigen or ligand. The target protein may be a protein from a cancer cell. The protein from a cancer cell may be an oncogenic protein, or a signaling mediator of an abnormal cellular proliferative pathway and its degradation decreases abnormal cell growth.

The target protein may be derived from a gene that has undergone an amplification, translocation, deletion, or inversion event which causes or is caused by a medical disorder. In certain aspects, the target protein has been post-translationally modified by one, or combinations, of phosphorylation, acetylation, acylation including propionylation and crotylation, N-linked glycosylation, amidation, hydroxylation, methylation, poly-methylation, O-linked glycosylation, pyroglutamoylation, myristovlation, farnesylation, geranylation, ubiquitination, sumoylation, or sulfation which causes or is caused by a medical disorder.

In some embodiments, the target protein is a protein that is not drugable in the classic sense in that it does not have a binding pocket or an active site that can be inhibited or otherwise bound, and cannot be casily allosterically controlled. In some embodiments, the target protein is a protein that is drugable in the classic sense, yet for therapeutic purposes, degradation of the protein is preferred to inhibition.

The target protein is recruited with a targeting domain. The targeting domain may be a ligand for the target protein. The targeting domain may bind the target protein in a non-covalent fashion.

The target protein may be any amino acid sequence to which a targeting domain of the fusion protein disclosed herein can be bound and subsequently degraded and causes a beneficial therapeutic effect in vivo. In some embodiments, the target protein is a non-endogenous peptide such as that from a pathogen or toxin. In some embodiments, the target protein can be an endogenous protein that mediates a disorder. The endogenous protein can be either the normal form of the protein or an aberrant form. For example, the target protein can be a mutant protein found in cancer cells, or a protein, for example, where a partial, or full, gain-of-function or loss-of-function is encoded by nucleotide polymorphisms. In some embodiments, the targeting domain of the fusion protein disclosed herein targets the aberrant form of the protein and not the normal form of the protein. In some embodiments, the target protein can mediate an inflammatory disorder or an immune disorder, including an auto-immune disorder. In some embodiments, the target protein is a non-endogenous protein from a virus, as non-limiting examples, HIV, HBV, HCV, RSV, HPV, CMV, flavivirus, pestivirus, coronavirus, noroviridae, etc. In some embodiments, the target protein is a non-endogenous protein from a bacteria, which may be for example, a gram positive bacteria, gram negative bacteria or other, and can be a drug-resistant form of bacteria. In some embodiments, the target protein is a non-endogenous protein from a fungus. In some embodiments, the target protein is a non-endogenous protein from a prion. In some embodiments, the target protein is a protein derived from a eukaryotic pathogen, for example a protist, helminth, etc.

In some embodiments, the target protein may mediate chromatin structure and function. The target protein may mediate an epigenetic action such as DNA methylation or covalent modification of histones. An example is histone deacetylase. In some embodiments, the target protein may be a bromodomain-containing protein, which are readers of lysine acetylation.

In some embodiments, the target protein may be a structural protein, receptor, enzyme, cell surface protein, a protein involved in apoptotic signaling, aromatase, helicase, mediator of a metabolic process (anabolism or catabolismantioxidant, protease, kinase, oxidoreductase, transferase, hydrolase, lyase, isomerase, ligase, enzyme regulator, signal transducer, structural molecule, binding activity (protein, lipid carbohydrate), cell motility protein, membrane fusion protein, cell communication mediator, regulator of biological processes, behavioral protein, cell adhesion protein, protein involved in cell death, or protein involved in transport (including protein transporter activity, nuclear transport, ion transporter, channel transporter, carrier activity, permease, secretase or secretion mediator, electron transporter, chaperone regulator, nucleic acid binding, transcription regulator, extracellular organization and biogenesis regulator, and translation regulator).

In some embodiments, the target protein is a modulator of a signaling cascade related to a known disease state. In some embodiments, the target protein mediates a disorder by a mechanism different from modulating a signaling cascade. Any protein in a cukaryotic system or a microbial system, including a virus, bacteria or fungus, as otherwise described herein, are targets for proteasomal degradation using the fusion proteins or ARMMs disclosed herein. The target protein may be a cukaryotic protein, and in some embodiments, a human protein.

In some embodiments, the target protein is RXR, DHFR, Hsp90, a kinase, HDM2, MDM2, BET bromodomain-containing protein, HDAC, IDH1, Mcl-1, human lysine methyltransferase, a nuclear hormone receptor, aryl hydrocarbon receptor (AHR), RAS, RAF, FLT, SMARC, KSR, NF2L, CTNB, CBLB, or BCL.

In some embodiments, the target protein is selected from: EGFR, FLT3, RAF1, SMRCA2, KSR1, NF2L2, CTNB1, CBLB, BCL6, and RASK.

In some embodiments, the target protein is an EGFR ligand, a FLT3 ligand, a RAF1 ligand, a SMRCA2 ligand, a KSR1 ligand, a NF2L2 ligand, a CTNB1 ligand, a CBLB ligand, a BCL6 ligand, or a RASK ligand.

The ARRDC1 fusion proteins or ARMMs disclosed herein may be used to treat a wide range of disease states and/or conditions, including any disease state and/or condition in which a protein is dysregulated and where a patient would benefit from the degradation of proteins.

For example, a target protein can be selected that is a known target for a human therapeutic, and the therapeutic can be used as the targeting domain when fused to an ARRDC1 protein or a variant thereof according to the present disclosure. These include proteins which may be used to restore function in a polygenic disease, including for example B7.1 and B7, TINFR1m, TNFR2, NADPH oxidase, Bcl2/Bax and other partners in the apoptosis pathway, C5a receptor, HMG-COA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, e.g., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuraminidasc, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VCAM, VLA-4 integrin, selectins, CD40/CD40L, neurokinins and receptors, inosine monophosphate dehydrogenase, p38 MAP Kinase, Ras/Raf/MER/ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors, adenosine receptors, adenosine kinase and AMP deaminase. purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-2/neu, telomerase inhibition, cytosolic phospholipaseA2 and EGF receptor tyrosine kinase. Additional protein targets include, for example, ccdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

In some embodiments, the target protein is derived from a kinase to which the targeting domain is capable of binding or binds including, but not limited to, a tyrosine kinase (e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYRO3, YES1, or ZAP70).

In some embodiments, the target protein is derived from a kinase to which the targeting domain is capable of binding or binds including, but not limited to, a serine/threonine kinase (e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAO1, TAO2, TGF-beta, TLK2, TSSK1, TSSK2, ULK1, or ULK2).

In some embodiments, the target protein is derived from a kinase to which the targeting domain is capable of binding or binds including, but not limited to a cyclin dependent kinase for example CDK1, CDK2, CDK3, CDK4, CDK5, CDK6, CDK7, CDK8, CDK9, CDK10, CDK11, CDK12, or CDK13.

In some embodiments, the target protein is derived from a kinase to which the targeting domain is capable of binding or binds including, but not limited to a leucine-rich repeat kinase (e.g., LRRK2).

In some embodiments, the target protein is derived from a kinase to which the targeting domain is capable of binding or binds including, but not limited to a lipid kinase (e.g., PIK3CA, PIK3CB) or a sphingosine kinase (e.g., S1P).

In some embodiments, the target protein is derived from a BET bromodomain-containing protein to which the targeting domain is capable of binding or binds including, but not limited to, ASH1L, ATAD2, BAZ1A, BAZ1B, BAZ2A, BAZ2B, BRD1, BRD2, BRD3, BRD4, BRD5, BRD6, BRD7, BRD8, BRD9, BRD10, BRDT, BRPF1, BRPF3, BRWD3, CECR2, CREBBP, EP300, FALZ, GCN5L2, KIAA1240, LOC93349, MLL, PB1, PCAF, PHIP, PRKCBP1, SMARCA2, SMARCA4, SP100, SP110, SP140, TAF1, TAF1L, TIF1a, TRIM28, TRIM33, TRIM66, WDR9, ZMYND11, and MLL4. In certain embodiments, a BET bromodomain-containing protein is BRD4.

In some embodiments, the target protein is derived from a nuclear protein to which the targeting domain is capable of binding or binds including, but not limited to, BRD2, BRD3, BRD4, Antennapedia Homcodomain Protein, BRCA1, BRCA2, CCAAT-Enhanced-Binding Proteins, histones, Polycomb-group proteins, High Mobility Group Proteins, Telomere Binding Proteins, FANCA, FANCD2, FANCE, FANCF, hepatocyte nuclear factors, Mad2, NF-kappa B. Nuclear Receptor Coactivators, CREB-binding protein, p55, p107, p130, Rb proteins, p53, c-fos, c-jun, c-mdm2, c-myc, and c-rel.

In some embodiments, the target protein is a member of the Retinoid X Receptor (RXR) family and the disorder treated is a neuropsychiatric or neurodegenerative disorder. In some embodiments. the target protein is a member of the Retinoid X Receptor (RXR) family and the disorder treated is schizophrenia.

In some embodiments, the target protein is dihydrofolate reductase (DHFR) and the disorder treated is cancer. In certain embodiments, the target protein is dihydrofolate reductase (DHFR) and the disorder treated is microbial. In some embodiments, the target protein is dihydrofolate reductase from bacillus anthracis (BaDHFR) and the disorder treated is anthrax. In some embodiments, the target protein is Heat Shock Protein 90 (HSP90) and the disorder treated is cancer. In some embodiments, the target protein is a kinase or phosphatase and the disorder treated is cancer. In some embodiments, the target protein is HDM2 and or MDM2 and the disorder treated is cancer. In some embodiments, the target protein is a BET bromodomain containing protein and the disorder treated is cancer. In some embodiments, the target protein is a lysine methyltransferase and the disorder treated is cancer. In some embodiments, the target protein belongs to the RAF family and the disorder treated is cancer. In some embodiments, the target protein belongs to the FKBP family and the disorder treated is an autoimmune disorder. In some embodiments, the target protein belongs to the FKBP family and the disorder treated is organ rejection. In some embodiments, the target protein belongs to the FKBP family and the compound is given prophylactically to prevent organ failure. In some embodiments, the target protein is an androgen receptor and the disorder treated is cancer. In some embodiments, the target protein is an estrogen receptor and the disorder treated is cancer. In some embodiments, the target protein is a viral protein and the disorder treated is a viral infection. In some embodiments, the target protein is a viral protein and the disorder treated is HIV. HPV, or HCV. In some embodiments, the target protein is an AP-1 or AP-2 transcription factor and the disorder treated is cancer. In some embodiments, the target protein is a HIV protease and the disorder treated is a HIV infection. In some embodiments, the target protein is a HIV integrase and the disorder treated is a HIV infection. In some embodiments, the target protein is a HCV protease and the disorder treated is a HCV infection. In some embodiments, the treatment is prophylactic and the target protein is a viral protein. In some embodiments, the target protein is a member of the histone deacetylase (HDAC) family and the disorder is a neurodegenerative disorder. In some embodiments, the target protein is a member of the histone deacetylase (HDAC) family and the disorder is Huntingon's, Parkinson's, Kennedy disease, amyotropic lateral sclerosis, Rubinstein-Taybi syndrome, or stroke.

In some embodiments, the target protein as referred to herein is named by the gene that expresses it. The person skilled in the art will recognize that when a gene is referred to as a target protein, the protein encoded by the gene is the target protein. Additional target proteins can be found in US20190076539A1, which is incorporated by reference herein in its entirety.

### Targeting Domains

In certain aspects, the targeting domain is a ligand which covalently or non-covalently binds to a target protein which has been selected for proteasomal degradation by the fusion protein. A targeting domain can be a small molecule or moiety (for example a peptide, nucleotide, antibody, antibody fragment, aptamer, biomolecule or other chemical structure) that binds to a target protein, and wherein the target protein is a mediator of disease in a host as described in detail previously. In some embodiments, the targeting domain is a non-enzymatic targeting domain. In some embodiments, the targeting domain comprises an antibody, a fragment of an antibody, or an antibody derivative. In some embodiments, the targeting domain comprises an antigen.

In some embodiments, the targeting domain binds to an endogenous protein which has been targeted for degradation as a means to achieve a therapeutic effect on the host. Illustrative targeting domains include: RXR ligands, DHFR ligands, ligands of MerTK, ligands of IDH1, ligands of Mcl-1, ligands of SMRCA2, ligands of EGFR, ligands of RAF, ligands of cRAF. In some embodiments, the targeting domain is an Aryl Hydrocarbon Receptor (AHR) ligand. Non-limiting examples of AHR ligands include: apigenin, SR1, LGC006, and analogues thereof. In some embodiments, the targeting domain is a MerTK or Mer targeting domain. In some embodiments, the targeting domain is an EGFR ligand. In some embodiments, the targeting domain is an EGRF ligand selected from Afatinib. Dacomitinib, Neratinib, Poziotinib, and Canertinib, or derivatives thereof. In some embodiments, the targeting domain is a FLT3 Ligand. In some embodiments, targeting domain is a FLT3 ligand selected from Tandutinib, Lestaurtinib. Sorafenib, Midostaurin, Quizartinib, and Crenolanib. In some embodiments, the Dabrafenib is an Ubc9 SUMO E2 ligase 5F6D targeting ligand. In some embodiments, the Dabrafenib is a Tank1 targeting ligand. In some embodiments, the targeting ligand is a SH2 domain of pp60 Src targeting ligand. In some embodiments, the Dabrafenib is a Sec7 domain targeting ligand. In some embodiments, the targeting domain is a Saposin-B targeting ligand. In some embodiments, the targeting domain is a Protein S100-A7 20WS targeting ligand. In some embodiments, the targeting domain is a Phospholipase A2 targeting ligand. In some embodiments, the targeting domain is a PHIP targeting ligand. In some embodiments, the targeting domain is a PDZ targeting ligand. In some embodiments, the targeting domain is a PARP15 targeting ligand. In some embodiments, the targeting domain is a PARP14 targeting ligand. In some embodiments, the targeting domain is a MTH1 targeting ligand. In some embodiments, the targeting domain is a mPGES-1 targeting ligand.

In some embodiments, the targeting domain is a FLAP-5-lipoxygenase-activating protein targeting ligand. In some embodiments, the targeting domain is a FA Binding Protein targeting ligand. In some embodiments, the targeting domain is a BCL2 targeting ligand. In some embodiments, the targeting domain is a NF2L2 targeting ligand. In some embodiments, the targeting domain is a CTNNB1 targeting ligand. In some embodiments, the targeting domain is a CBLB targeting ligand. In some embodiments, the targeting domain is a BCL6 targeting ligand. In some embodiments, the targeting domain is a RASK targeting ligand. In some embodiments, the targeting domain is a TNIK targeting ligand. In some embodiments, the targeting domain is a MEN1 targeting ligand. In some embodiments, the targeting domain is a PI3Ka targeting ligand. In some embodiments, the targeting domain is an IDO1 targeting ligand. In some embodiments, the targeting domain is an MCL1 targeting ligand. In some embodiments, the targeting domain is a PTPN2 targeting ligand. In some embodiments, the targeting domain is a HER2 targeting ligand. In some embodiments, the targeting ligand is an EGFR targeting ligand. In one embodiment the EGFR targeting ligand is selected from erlotinib (Tarceva), gefitinib (Iressa), afatinib (Gilotrif), rocilctinib (CO-1686), osimertinib (Tagrisso), olmutinib (Olita), naquotinib (ASP8273), nazartinib (EGF816), PF-06747775 (Pfizer), icotinib (BPI-2009), neratinib (HKI-272: PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer). The linker can be placed on these targeting ligands in any location that does not interfere with the ligands binding to EGFR. In some embodiments, the EGFR targeting ligand binds the L858R mutant of EGFR. In some embodiments, the EGFR targeting ligand binds the T790M mutant of EGFR. In some embodiments, the EGFR targeting ligand binds the C797G or C797S mutant of EGFR. In some embodiments, the EGFR targeting ligand is selected from erlotinib, gefitinib, afatinib, neratinib, and dacomitinib and binds the L858R mutant of EGFR. In some embodiments, the EGFR targeting ligand is selected from osimertinib, rociletinib, olmutinib, naquotinib, nazartinib, PF-06747775, Icotinib, Neratinib, Avitinib, Tarloxotinib, PF-0645998, Tesevatinib, Transtinib, WZ-3146, WZ8040, and CNX-2006 and binds the T790M mutant of EGFR. In some embodiments, the EGFR targeting ligand is EA1045 and binds the C797G or C797S mutant of EGFR.

In some embodiments, the target protein and targeting domain pair are chosen by screening a library of ligands. Such a screening is exemplified in “Kinase Inhibitor Profiling Reveals Unexpected Opportunities to Inhibit Disease-Associated Mutant Kinases” by Duong-Ly et al.; Cell Reports 14, 772-781 Feb. 2, 2016.

In some embodiments, the target protein and targeting domain pair are discovered by screening promiscuous kinase binding ligands for context-specific degradation.

Additional targeting domains can be found in US20190076539A1, which is incorporated by reference herein in its entirety.

### Indications

In some aspects, the fusion proteins or ARMMs described herein may also be used in methods of treatment of a subject in need thereof, comprising administering to the subject in need thereof a therapeutically effective amount of the fusion proteins or ARMMs described herein. Treatment can be therapeutic, prophylactic or preventative. Treatment encompasses alleviation, reduction, or prevention of at least one aspect or symptom of a disease and encompasses prevention or cure of the diseases described herein.

The disease may be an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. Abnormal cellular proliferation, notably hyperproliferation, can occur as a result of a wide variety of factors, including genetic mutation, infection, exposure to toxins, autoimmune disorders, and benign or malignant tumor induction. There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma. Other hyperproliferative cell disorders include blood vessel proliferation disorders, fibrotic disorders, autoimmune disorders, graft-versus-host rejection, tumors and cancers.

In some embodiments, the proliferative disorder is a cancer. The cancer may be squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases: multiple myeloma, sarcomas, including Ewing's sarcoma, hemangiosarcoma. Kaposi's sarcoma, liposarcoma, myosarcomas, peripheral neuroepithelioma. synovial sarcoma, gliomas, astrocytomas, oligodendrogliomas, ependymomas, gliobastomas, neuroblastomas, ganglioncuromas, gangliogliomas, medulloblastomas, pineal cell tumors, meningiomas, meningcal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor or teratocarcinomas, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, or Philadelphia chromosome positive CML.

The disease state or condition may be a disease caused by a microbial agent or other exogenous agent such as a virus (as non-limiting examples, HIV, HBV, HCV, HSV, HPV, RSV, CMV, Ebola, Flavivirus, Pestivirus, Rotavirus, Influenza, Coronavirus, EBV, viral pneumonia, drug-resistant viruses, Bird flu, RNA virus, DNA virus, adenovirus, poxvirus, Picornavirus, Togavirus, Orthomyxovirus, Retrovirus or Hepadnovirus), bacteria (Gram-negative, Gram-positive, fungus, protozoa, helminth, worms, prion, parasite, or other microbe or may be a disease state, which is caused by overexpression of a protein, which leads to a disease state and/or condition.

Further disease states or conditions which may be treated by ARRDC1 fusion proteins or ARMMs according to the present disclosure include Alzheimer's disease, Amyotrophic lateral sclerosis (Lou Gehrig's disease), Anorexia nervosa, Anxiety disorder, Atherosclerosis, Attention deficit hyperactivity disorder, Autism, Bipolar disorder, Chronic fatigue syndrome, Chronic obstructive pulmonary disease, Crohn's disease, Coronary heart disease, Dementia, Depression, Diabetes mellitus type 1, Diabetes mellitus type 2, Epilepsy, Guillain-Barré syndrome, Irritable bowel syndrome, Lupus, Metabolic syndrome, Multiple sclerosis, Myocardial infarction, Obesity, Obsessive-compulsive disorder, Panic disorder, Parkinson's disease, Psoriasis, Rheumatoid arthritis, Sarcoidosis, Schizophrenia, Stroke, Thromboangiitis obliterans, Tourette syndrome, Vasculitis.

Still additional disease states or conditions which can be treated by ARRDC1 fusion proteins or ARMMs according to the present disclosure include aceruloplasminemia, Achondrogenesis type II, achondroplasia, Acrocephaly, Gaucher disease type 2, acute intermittent porphyria, Canavan disease, Adenomatous Polyposis Coli, ALA dehydratase deficiency, adenylosuccinate lyase deficiency, Adrenogenital syndrome, Adrenoleukodystrophy, ALA-D porphyria, ALA dehydratase deficiency, Alkaptonuria, Alexander disease, Alkaptonuric ochronosis, alpha 1-antitrypsin deficiency, alpha-1 proteinase inhibitor, emphysema, amyotrophic lateral sclerosis Alstrom syndrome, Alexander disease, Amelogenesis imperfecta, ALA dehydratase deficiency, Anderson-Fabry disease, androgen insensitivity syndrome, Anemia Angiokeratoma Corporis Diffusum, Angiomatosis retinac (von Hippel-Lindau disease) Apert syndrome, Arachnodactyly (Marfan syndrome), Stickler syndrome, Arthrochalasis multiplex congenital (Ehlers-Danlos syndrome #arthrochalasia type) ataxia telangiectasia, Rett syndrome, primary pulmonary hypertension, Sandhoff disease, neurofibromatosis type II, Beare-Stevenson cutis gyrata syndrome, Mediterranean fever, familial, Benjamin syndrome, beta-thalassemia, Bilateral Acoustic Neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dubé syndrome, Brittle bone disease (osteogenesis imperfecta), Broad Thumb-Hallux syndrome (Rubinstein-Taybi syndrome), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, Cardiomyopathy (Noonan syndrome), Cri du chat, CAVD (congenital absence of the vas deferens), Caylor cardiofacial syndrome (CBAVD), CEP (congenital erythropoictic porphyria), cystic fibrosis, congenital hypothyroidism, Chondrodystrophy syndrome (achondroplasia), otospondylomegaepiphyscal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoictic porphyria, Congenital heart disease, Methemoglobinemia/Congenital methacmoglobinacmia, achondroplasia, X-linked sideroblastic anemia, Connective tissue disease, Conotruncal anomaly face syndrome, Cooley's Anemia (beta-thalassemia), Copper storage disease (Wilson's disease), Copper transport disease (Menkes disease), hereditary coproporphyria, Cowden syndrome, Craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare-Stevenson cutis gyrata syndrome, primary hyperoxaluria, spondyloepimetaphyscal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria Erythroid 5-aminolevulinate synthetase deficiency, Erythropoietic porphyria, erythropoictic protoporphyria, erythropoictic uroporphyria, Friedreich's ataxia-familial paroxysmal polyscrositis, porphyria cutanea tarda, familial pressure sensitive neuropathy, primary pulmonary hypertension (PPH), Fibrocystic disease of the pancreas, fragile X syndrome, galactosemia, genetic brain disorders, Giant cell hepatitis (Neonatal hemochromatosis), Gronblad-Strandberg syndrome (pseudoxanthoma elasticum), Gunther disease (congenital erythropoictic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, Kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, Movement disorders, Mowat-Wilson syndrome, cystic fibrosis, Muenke syndrome, Multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondylocpimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, Thyroid disease, Tomaculous neuropathy (hereditary neuropathy with liability to pressure palsies), Treacher Collins syndrome, Triplo X syndrome (triple X syndrome), Trisomy 21 (Down syndrome), Trisomy X, VHL syndrome (von Hippel-Lindau disease), Vision impairment and blindness (Alstrom syndrome), Vrolik disease, Waardenburg syndrome, Warburg Sjo Fledelius Syndrome, Weissenbacher-Zweymüller syndrome, Wolf-Hirschhorn syndrome, Wolff Periodic disease, Weissenbacher-Zweymüller syndrome and Xeroderma pigmentosum, among others.

### Combination Therapy

The ARRDC1 fusion proteins or ARMMs disclosed herein may be used in an effective amount alone or in combination to treat a subject with a disorder as described herein.

The ARRDC1 fusion proteins or ARMMs disclosed herein may be used in an effective amount alone or in combination with another ARRDC1 fusion protein or ARMM of the present disclosure or another bioactive agent to treat a subject with a disorder as described herein.

In some embodiments, the ARRDC1 fusion protein or ARMM of the present disclosure and the bioactive agent are administered in a manner that they are active in vivo during overlapping time periods, for example, have time-period overlapping Cmax, Tmax, AUC or other pharmacokinetic parameter. In some embodiments, the ARRDC1 fusion protein or ARMM of the present disclosure and the bioactive agent are administered to a host in need thereof that do not have overlapping pharmacokinetic parameter, however, one has a therapeutic impact on the therapeutic efficacy of the other.

In some embodiments, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

PD-1 inhibitors that blocks the interaction of PD-1 and PD-L1 by binding to the PD-1 receptor, and in turn inhibit immune suppression include, for example, nivolumab (Opdivo), pembrolizumab (Keytruda), pidilizumab, AMP-224 (AstraZeneca and MedImmune), PF-06801591 (Pfizer), MEDI0680 (AstraZeneca), spartalizumab (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atczolizumab (Tecentriq), durvalumab (AstraZeneca and MedImmune), KN035 (Alphamab), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and MedImmune), AGEN1884 and AGEN2041 (Agenus). LAG-3 checkpoint inhibitors, include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). Examples of of a TIM-3 inhibitors include sabatolimab (Novartis) and TSR-022 (Tesaro).

In some embodiments, the bioactive agent is an ALK inhibitor. Examples of ALK inhibitors include but are not limited to Crizotinib. Alectinib, ceritinib, TAE684 (NVP-TAE684), GSK1838705A, AZD3463, ASP3026, PF-06463922, entrectinib (RXDX-101), and AP26113.

In some embodiments, the bioactive agent is an EGFR inhibitor. Examples of EGFR inhibitors include erlotinib (Tarceva), gefitinib (Iressa), afatinib (Gilotrif), rociletinib (CO-1686), osimertinib (Tagrisso), olmutinib (Olita), naquotinib (ASP8273), nazartinib (EGF816), PF-06747775 (Pfizer). icotinib (BPI-2009), neratinib (HKI-272: PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer).

In some embodiments, the bioactive agent is an HER-2 inhibitor. Examples of HER-2 inhibitors include trastuzumab, lapatinib, ado-trastuzumab emtansine, and pertuzumab. In some embodiments, the bioactive agent is a CD20 inhibitor. Examples of CD20 inhibitors include obinutuzumab, rituximab, fatumumab, ibritumomab, tositumomab, and ocrelizumab. In some embodiments, the bioactive agent is a JAK3 inhibitor. Examples of JAK3 inhibitors include tasocitinib.

In one embodiment, the bioactive agent is a BCL-2 inhibitor. Examples of BCL-2 inhibitors include venetoclax. ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenyl sulfanylbutan-2-yl]amino]-3-nitrophenyl]sulfonylbenzamide) (navitoclax), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3 ((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide). Apogossypolone (ApoG2), HA14-1, AT101, sabutoclax, gambogic acid, or G3139 (Oblimersen).

In some embodiments, the bioactive agent is a kinase inhibitor. In some embodiments, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

Examples of PI3 kinase inhibitors include but are not limited to Wortmannin, demethoxyviridin. perifosine, idelalisib, Pictilisib, Palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)-N1-[4-Methyl-5-[2-(2,2,2-trifluoro-1,1-dimethylethyl)-4-pyridinyl]-2-thiazolyl]-1,2-pyrrolidinedicarboxamide). GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((+)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid). TGR-1202/RP5264. GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz). AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methyl sulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl] acetate (also known as sonolisib)), LY294002, AZD8186, PF-4989216, pilaralisib, GNE-317, PI-3065, PI-103, NU7441 (KU-57788), HS 173 VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422).

Examples of BTK inhibitors include ibrutinib (also known as PCI-32765) (Imbruvica™) (1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors include, for example. Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), RO9021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (sec Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

In some embodiments, the bioactive agent is a MEK inhibitor. MEK inhibitors are well known, and include, for example, trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)-N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEA1 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy[-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H, 8H)-dione), MEK 162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methyl sulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide), U0126-EtOH, PD184352 (CI-1040), GDC-0623, BI-847325, cobimetinib, PD98059, BIX 02189, BIX 02188, binimetinib, SL-327, TAK-733, PD318088.

In some embodiments, the bioactive agent is a Raf inhibitor. Raf inhibitors are known and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), Sorafenib N-Oxide (4-[4-[[[[4-Chloro-3 (trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, SB590885, ZM336372, GW5074, TAK-632, CEP-32496, LY3009120, and GX818 (Encorafenib).

In some embodiments, the bioactive agent is an AKT inhibitor, including but not limited to, MK-2206, GSK690693, Perifosine, (KRX-0401), GDC-0068, Triciribine, AZD5363, Honokiol, PF-04691502, and Miltefosine, a FLT-3 inhibitor, including but not limited to, P406, Dovitinib, Quizartinib (AC220), Amuvatinib (MP-470), Tandutinib (MLN518), ENMD-2076, and KW-2449, or a combination thereof.

In some embodiments, the bioactive agent is an mTOR inhibitor. Examples of mTOR inhibitors include but are not limited to rapamycin and its analogs, everolimus (Afinitor), temsirolimus. ridaforolimus, sirolimus, and deforolimus. Examples of MEK inhibitors include but are not limited to tametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinob (6-(4-bromo-2-chloroanilino)-7-fluoro-N-(2-hydroxyethoxy)-3-methylbenzimidazole-5-carboxamide), pimasertib/AS703026/MSC1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol) (cobimetinib). refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy[-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2 yl)methyl)benzamide), or AZD8330 (2-((2-fluoro-4-iodophenyl)amino)-N-(2-hydroxyethoxy)-1, 5-dimethyl-6-oxo-1,6-dihydropyridine-3-carboxamide).

In some embodiments, the bioactive agent is a RAS inhibitor. Examples of RAS inhibitors include but are not limited to Reolysin and siG12D LODER. In some embodiments, the bioactive agent is an HSP inhibitor. HSP inhibitors include but are not limited to Geldanamycin or 17-N-Allylamino-17-demethoxygeldanamycin (17AAG), and Radicicol.

Additional bioactive compounds include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an aurora kinase inhibitor, a PIK-1 modulator, an HDAC inhbitor, a c-MET inhibitor, a PARP inhibitor, a Cdk inhibitor, an IGFR-TK inhibitor, an anti-HGF antibody, a focal adhesion kinase inhibitor, a Map kinase kinase (mek) inhibitor, a VEGF trap antibody, pemetrexed, panitumumab, amrubicin, oregovomab, Lep-ctu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdRiKRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethyl stilbestrol), estradiol, estrogen, conjugated estrogen, bevacizumab, IMC-IC11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SUI11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustinc, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoctin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginasc, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In some embodiments, the bioactive agent is selected from, but are not limited to, Imatinib mesylate (Gleevac®), Dasatinib (Sprycel®), Nilotinib (Tasigna®), Bosutinib (Bosulif®), Trastuzumab (Herceptin®), trastuzumab-DM1, Pertuzumab (Perjeta™), Lapatinib (Tykerb®), Gefitinib (Iressa®), Erlotinib (Tarceva®), Cetuximab (Erbitux ®), Panitumumab (Vectibix®), Vandetanib (Caprelsa®), Vemurafenib (Zelboraf®), Vorinostat (Zolinza®), Romidepsin (Istodax®), Bexarotene (Tagretin®), Alitretinoin (Panretin®), Tretinoin (Vesanoid®), Carfilizomib (Kyprolis™), Pralatrexate (Folotyn ®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

In some aspects, the bioactive agent is an anti-inflammatory agent, a chemotherapeutic agent, a radiotherapeutic, an additional therapeutic agent, or an immunosuppressive agent.

Suitable chemotherapeutic bioactive agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: Vincristine (Oncovin®) or liposomal vincristine (Marqibo®), Daunorubicin (daunomycin or Cerubidine®) or doxorubicin (Adriamycin®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Methotrexate, Cyclophosphamide (Cytoxan®), Prednisone, Dexamethasone (Decadron), imatinib (Gleevec®), dasatinib (Sprycel®), nilotinib (Tasigna®), bosutinib (Bosulif®), and ponatinib (Iclusig™). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Additional therapeutic agents that can be administered in combination with a degronimer disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacctuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991, ribociclib (LEE011), amebaciclib (LY2835219), HDM201, fulvestrant (Faslodex), exemestane (Aromasin), PIM447, ruxolitinib (INC424), BGJ398, necitumumab, pemetrexed (Alimta), and ramucirumab (IMC-1121B).

In some aspects, the ARRDC1 fusion protein or ARMM disclosed herein is administered in combination with an anti-infective agent, for example but not limited to an anti-HIV agent, anti-HCV agent, anti-HBV agent, or other anti-viral or anti-bacterial agent. In one embodiment, the anti-HIV agent can be, but is not limited to, for example, a nucleoside reverse transcriptase inhibitor (NRTI), other non-nuclocoside reverse transcriptase inhibitor, protease inhibitor, fusion inhibitor, among others. Nucleoside/Nucleotide Reverse Transcriptase Inhibitors (NRTIs) include, but are not limited to, Abacavir or ABC (Ziagen), Didanosine or ddl (Videx), Emtricitabine or FTC (Emtriva), Lamivudine or 3TC (Epivir), ddC (zalcitabine), Stavudine or d4T (Zerit), Tenofovircor TDF (Viread), D-D4FC (Reverset), and Zidovudine or AZT or ZDV (Retrovir). Non-nucleoside Reverse Transcriptase Inhibitors (NNRTIs) include, but are not limited to, Delavirdine (Rescriptor), Efavirenz (Sustiva), Etravirine (Intelence), Nevirapine (Viramune), and Rilpivirine (Edurant). Anti-HIV Protease Inhibitors (PIs) include, but are not limited to, Atazanavir or ATV (Reyataz), Darunavir or DRV (Prezista), Fosamprenavir or FPV (Lexiva), Indinavir or IDV (Crixivan), Lopinavir+ritonavir, or LPV/r (Kaletra), Nelfinavir or NFV (Viracept), Ritonavir or RTV (Norvir), Saquinavir or SQV (Invirase), Tipranavir, or TPV (Aptivus), Cobicistat (Tybost), Atazanavir+cobicistat, or ATV/COBI (Evotaz), Darunavir+cobicistat, or DRV/COBI (Prezcobix). Anti-HIV Fusion Inhibitors include, but are not limited to, Enfuvirtide or ENF or T-20 (Fuzcon). Anti-HIV also include, but are not limited to, Maraviroc or MVC (Selzentry). Anti-HIV Integrase Inhibitors include, but are not limited to Dolutegravir (Tivicay), Elvitegravir (Vitekta), Raltegravir (Isentress). Anti-HIV combinations agents include Abacavir+Dolutegravir+lamivudine, or ABC/DTG/3TC (Triumeq), Abacavir+lamivudine or ABC/3TC (Epzicom), Abacavir+lamivudine+zidovudine, or ABC/3TC/ZDV (Trizivir), Efavirenz+emtricitabine+tenofovir or EFV/FTC/TDF (Atripla, Tribuss), elvitegravir, cobicistat, emtricitabine, tenofovir alafenamide or EVG/COBI/FTC/TAF or ECF/TAF (Genvoya; (Stribild), emtricitabine+rilpivirine+tenofovir or FTC/RPV/TAF (Odefsey); Emtricitabine+rilpivirine+tenofovir or FTC/RPV/TDF (Complera), Emtricitabine+tenofovir or TAF/FTC (Descovy), emtricitabine and tenofovir disoproxil fumarate (Truvada), and Lamivudine+zidovudine or 3TC/ZDV (Combivir). Other anti-HIV compounds include, but are not limited to Racivir, L-FddC, L-FD4C, SQVM (Saquinavir mesylate), IDV (Indinavir), SQV (Saquinavir), APV (Amprenavir), LPV (Lopinavir), fusion inhibitors such as T20, among others, fuseon and mixtures thereof, including anti-HIV compounds presently in clinical trials or in development.

Other anti-HIV agents which may be used in co-administration with the disclosed ARRDC1 fusion protein or ARMM according to the present disclosed herein may be selected from the group consisting of nevirapine (BI-R6-587), delavirdine (U-90152S/T), efavirenz (DMP-266), UC-781 (N-[4-chloro-3-(3-methyl-2-butenyloxy)phenyl]-2methyl3-furancarbothiamide), etravirinc (TMC125), Trovirdine (Ly300046·HCl), HI-236, HI-240, HI-280, HI-281, rilpivirine (TMC-278), MSC-127, HBY 097, DMP266, Baicalin (TJN-151) ADAM-II (Methyl 3′,3′-dichloro-4′,4″-dimethoxy-5′,5″-bis(methoxycarbonyl)-6,6-diphenylhexenoate), Methyl 3-Bromo-5-(1-5-bromo-4-methoxy-3-(methoxycarbonyl)phenyl)hept-1-enyl)-2-methoxybenzoate (Alkenyldiarylmethane analog, Adam analog), (5-chloro-3-(phenylsulfinyl)-2′-indolecarboxamide), AAP-BHAP (U-104489 or PNU-104489), Capravirine (AG-1549, S-1153), atevirdine (U-87201E), aurin tricarboxylic acid (SD-095345), 1-[(6-cyano-2-indolyl)carbonyl]-4-[3-(isopropylamino)-2-pyridinyl]piperazine, 1-[5-[N-(methyl)methylsulfonylamino]-2-indolylcarbonyl-4-[3-(isopropylamino)-2-pyridinyl]piperazine, 1-[3-(Ethylamino)-2-[pyridinyl]-4-[(5-hydroxy-2-indolyl)carbonyl]piperazine, 1-[(6-Formyl-2-indolyl)carbonyl]-4-[3-(isopropylamino)-2-pyridinyl]piperazine, 1-[[5-(Methylsulfonyloxy)-2-indoyly)carbonyl]-4-[3-(isopropylamino)-2-pyridinyl]piperazine, U88204E, Bis(2-nitrophenyl)sulfone (NSC 633001), Calanolide A (NSC675451), Calanolide B, 6-Benzyl-5-methyl-2-(cyclohexyloxy)pyrimidin-4-one (DABO-546), DPC 961, E-EBU, E-EBU-dm, E-EPSeU, E-EPU, Foscarnet (Foscavir), HEPT (1-[(2-Hydroxyethoxy)methyl]-6-(phenylthio)thymine), HEPT-M (1-[(2-Hydroxyethoxy)methyl]-6-(3-methylphenyl)thio)thymine), HEPT-S (1-[(2-Hydroxyethoxy)methyl]-6-(phenylthio)-2-thiothymine), Inophyllum P, L-737,126, Michellamine A (NSC650898), Michellamine B (NSC649324), Michellamine F, 6-(3,5-Dimethylbenzyl)-1-[(2-hydroxyethoxy)methyl]-5-isopropyluracil, 6-(3,5-Dimethylbenzyl)-1-(ethyoxymethyl)-5-isopropyluracil, NPPS, E-BPTU (NSC 648400), Oltipraz (4-Methyl-5-(pyrazinyl)-3H-1,2-dithiole-3-thione), N-{2-(2-Chloro-6-fluorophenethyl]-N′-(2-thiazolyl)thiourea (PETT Cl, F derivative), N-{2-(2,6-Difluorophenethyl]-N′-[2-(5-bromopyridyl)]thiourea {PETT derivative). N-{2-(2,6-Difluorophenethyl]-N′-[2-(5-methylpyridyl]thiourea {PETT Pyridyl derivative), N-[2-(3-Fluorofuranyl)ethyl]-N′-[2-(5-chloropyridyl)]thiourea, N-[2-(2-Fluoro-6-ethoxyphenethyl)]-N′-[2-(5-bromopyridyl)]thiourea, N-(2-Phenethyl)-N′-(2-thiazolyl)thiourea (LY-73497), L-697,639, L-697,593, L-697,661, 342-(4,7-Difluorobenzoxazol-2-yl)ethyl}-5-ethyl-6-methyl(pypridin-2(1H)-thione (2-Pyridinone Derivative), 3-[[(2-Methoxy-5,6-dimethyl-3-pyridyl)methyl]amine]-5-ethyl-6-methyl(pypridin-2(1H)-thione, R82150, R82913, R87232, R88703, R89439 (Loviride), R90385, S-2720, Suramin Sodium, TBZ (Thiazolobenzimidazole, NSC 625487), Thiazoloisoindol-5-one, (+)(R)-9b-(3,5-Dimethylphenyl-2,3-dihydrothiazolo[2,3-a]isoindol-5 (9bH)-one, Tivirapine (R86183), UC-38 and UC-84, among others.

In other aspects, the disclosed ARRDC1 fusion protein or ARMM when used to treat an HCV infection can be administered in combination with another anti-HCV agent. Anti-HCV agents are known in the art. To date, a number of fixed dose drug combinations have been approved for the treatment of HCV. Harvoni® (Gilead Sciences, Inc.) contains the NS5A inhibitor ledipasvir and the NS5B inhibitor sofosbuvir. Technivie™ (AbbVie, Inc.) is a fixed-dose combination containing ombitasvir, an NS5A inhibitor; paritaprevir, an NS3/4A protease inhibitor; and ritonavir, a CYP3A inhibitor. Daklinza™ (daclatasvir, Bristol-Myers Squibb) is a HCV NS5A inhibitor indicated for use with sofosbuvir for the treatment of chronic genotype 3 infection. Zepatier™ (Merck & Co.) has recently been approved for the treatment of chronic HCV genotypes 1 and 4. Zepatier™ is a fixed-dose combination product containing elbasvir, an HCV NS5A inhibitor, and grazoprevir, an HCV NS3/4A protease inhibitor. Zepatier™ is indicated with or without ribavirin. Epclusa® (Gilead Sciences, Inc.) is a fixed-dose combination tablet containing sofosbuvir and velpatasvir.

In some embodiments, the additional therapy is a monoclonal antibody (MAb). Some MAbs stimulate an immune response that destroys cancer cells. Similar to the antibodies produced naturally by B cells, these MAbs may “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels.

Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

In some aspects, the bioactive agent is an immunosuppressive agent. The immunosuppressive agent can be a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g. ridaforolimus, azathioprine, campath 1H, a S1P receptor modulator, e.g. fingolimod or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3 ®), Prednisone, ATGAM®, THYMOGLOBULIN®, Brequinar Sodium, OKT4, T10B9.A-3A, 33B3.1, 15-deoxyspergualin, tresperimus, Leflunomide ARAVA®, CTLAI-Ig, anti-CD25, anti-IL2R. Basiliximab (SIMULECT®), Daclizumab (ZENAPAX®), mizorbine, methotrexate, dexamethasone, ISAtx-247, SDZ ASM 981 (pimecrolimus, Elidel®), CTLA4lg (Abatacept), belatacept, LFA3lg, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

Additional bioactive agents can be found in US20190076539A1, which is incorporated by reference herein in its entirety.

### Methods for Preparation

The ARRDC1 fusion proteins disclosed herein can be prepared by methods known by those skilled in the art. For example, the ARRDC1 fusion proteins can be produced genetically or chemically. Provided herein are methods for preparing an arrestin domain-containing protein 1 (ARRDC1)-mediated microvesicle (ARMM), comprising (a) culturing a host cell comprising a recombinant expression construct encoding the ARRDC1 fusion protein as disclosed herein under the control of a heterologous promoter in a medium; and (b) collecting the ARMMs from the medium.

In some embodiments, the host cell has a reduced expression level of ubiquitin ligase as compared to a host cell of wildtype. In some embodiments, the host cell is genetically engineered to have a reduced expression level of ubiquitin ligase as compared to a wildtype cell. In some embodiments, the expression of the ubiquitin ligase in the host cell is reduced or inhibited by anti-sense oligonucleotide (ASN) technology, RNA interference (RNAi) technology such as small interefering RNA (siRNA) or short hairpain RNA (shRNA) or by gene editing tools known by those skilled in the art, such as CRISPR/Cas9 or zinc finger nuclease technology. In some embodiments, the expression of the ubiquitin ligase in the host cell is reduced or inhibited by a small molecular inhibitor known by those skilled in the art. In some embodiments, the activity of the ubiquitin ligase in the host cell is reduced or inhibited. In some embodiments, the activity of the ubiquitin ligase in the host cell is reduced or inhibited by a small molecular inhibitor known by those skilled in the art. In some embodiments, the amount or activity of the ubiquitin ligase expressed in the host cell is so minimal that it does not substantially interfer with the production of ARMMs in the host cell. In some embodiments, the host cell lacks the ubiquitin ligase. That is, the expression level of the ubiquitin ligase cannot be detected by tools available in the art. In some embodiments, the host cell lacks at least one ubiquitin ligase. In some embodiments, the host cell lacks one or more ubiquitin ligase. The ubiquitin ligase may be selected from the group consisting of HECW2, HECW1, WWPL, WWP2, NEDD4-1, NEDD4-2, NEDD4L, SMURFL, SMURF2, SMURF1, ITCH, NEDL1 and NEDL2.

In some embodiments, the host cell has a reduced expression level of the target protein as compared to a host cell of wildtype. In some embodiments, the host cell is genetically engineered to have a reduced expression level of the target protein as compared to a wildtype cell. In some embodiments, the expression of the target protein in the host cell is reduced or inhibited by anti-sense oligonucleotide (ASN) technology, RNA interference (RNAi) technology such as small interefering RNA (siRNA) or short hairpain RNA (shRNA) or by gene editing tools known by those skilled in the art, such as CRISPR/Cas9 or zinc finger nuclease technology. In some embodiments, the expression of the the target protein in the host cell is reduced or inhibited by a small molecular inhibitor known by those skilled in the art. In some embodiments, the amount of the target protein expressed in the host cell is so minimal that it does not substantially interfer with the production of ARMMs in the host cell. In some embodiments, the host cell lacks the target protein. That is, the expression level of the target protein cannot be detected by tools available in the art.

### Methods for Degradation of Protein and Alternation of Gene Expression

In some aspects, the present disclosure provides methods for degrading a target protein in a target cell, comprising contacting the target cell with an ARMM disclosed herein. The target protein can be any of the target proteins as disclosed herein.

In some aspects, the present disclosure provides methods for altering expression of at least one gene, comprising contacting the target cell with an ARMM disclosed herein. The at least one gene can be any gene that is regulated, directly or indirectly, by the target protein that is degraded by the ARMM disclosed herein.

By contacting the ARMM to the target cell, there is no need to worry about cell permeability which is often the problem of direct administration of fusion protins. Moreover, there is a reduced risk of immune reaction against the fusion proteins when delivered in ARMMs as opposed to direct administration. In some embodiments, the target cell is a mammalian cell. In some embodiments, the target cell is a human cell. In some embodiments, the target cell is a stem cell. In some embodiments, the target cell is a cell in vitro or ex vivo, and the method comprises administering the ARMM to the cell in vitro, or co-culturing the target cell with the microvesicle-producing cell in vitro. In some embodiments, the target cell is a cell in a subject, and the method comprises administering the microvesicle or the microveside-producing cell to the subject. In some embodiments, the subject is a mammalian subject. In some embodiments, the subject is a human subject. In some embodiments, the target cell is a pathological cell. In some embodiments, the target cell is a cancer cell. In some embodiments, the microvesicle includes a targeting agent that selectively binds an antigen of the target cell. In some embodiments, the antigen of the target cell is a cell surface antigen. In some embodiments, the targeting agent is a membrane-bound immunoglobulin, an integrin, a receptor, a receptor ligand, an aptamer, a small molecule, or a fragment thereof.

All references and publications cited herein are hereby incorporated by reference.

The following examples are provided to further illustrate some embodiments of the present invention, but are not intended to limit the scope of the invention: it will be understood by their exemplary nature that other procedures, methodologies, or techniques known to those skilled in the art can alternatively be used.

### EXAMPLES

### Example 1: Degradation of mCherry by Recruitment to ARRDC1 via Fusion with Single Domain Antibody (sdAb)

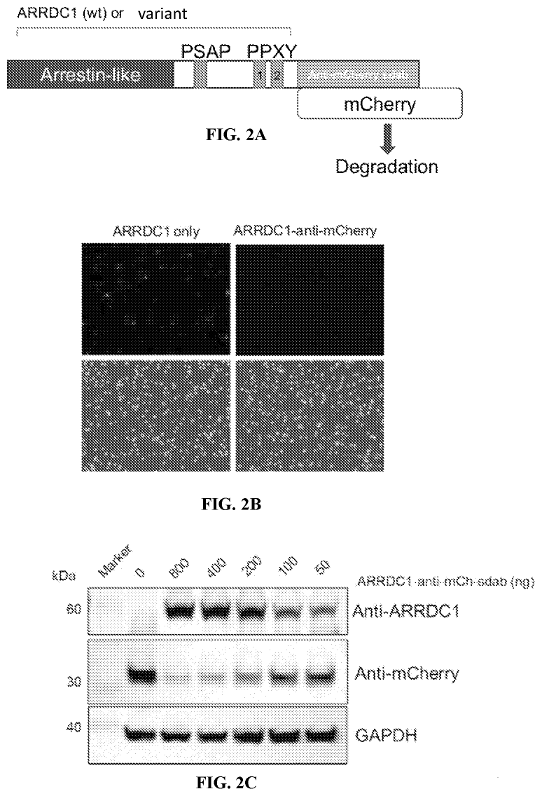

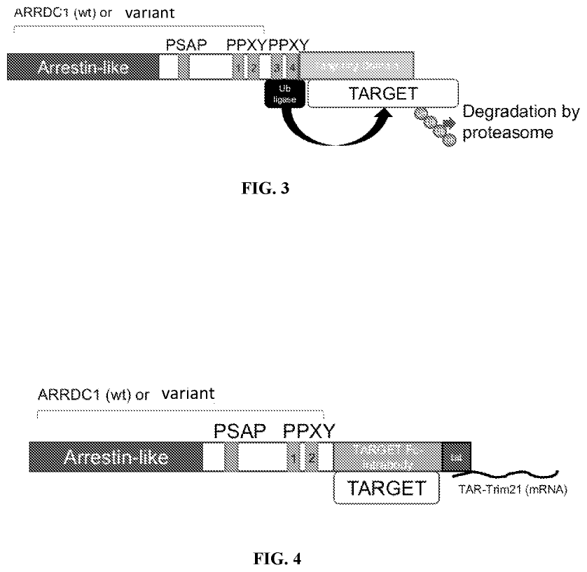

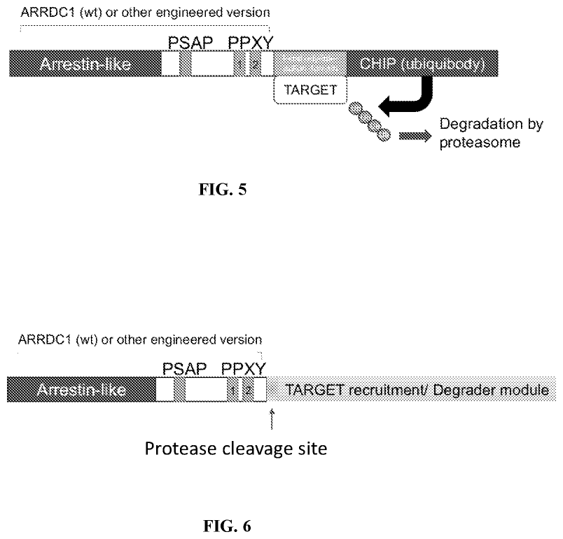

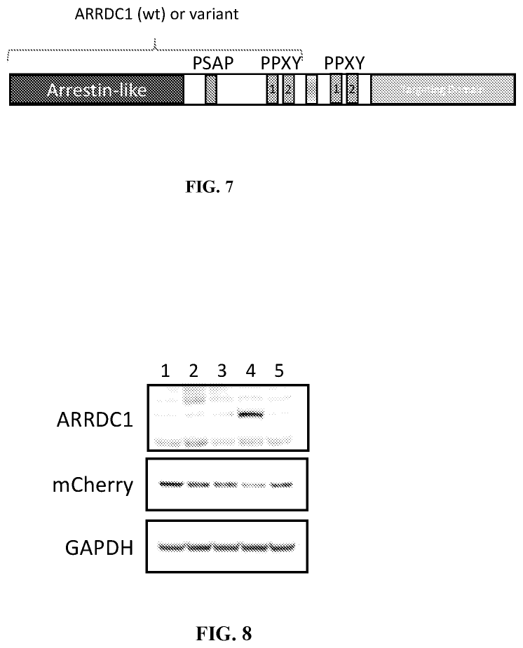

This example demonstrates a decrease in mCherry protein levels by using the ARRDC1 fusion protein comprising the ARRDC1 and a single domain antibody that binds mCherry (FIG. 2A). 293T cells were co-transfected with 50 ng mCherry expression construct and a construct encoding the ARRDC1 fusion protein. Cells were imaged for fluorescence signal 72 hours after transfection (FIG. 2B). The lysates were harvested and analyzed by western blotting (FIG. 2C). The results show that the fusion protein can successfully facilitate degradation of mCherry in a dose-dependent manner. The decrease of mCherry protein level correlates with the increase of ARRDC1 fusion protein (FIG. 2C).

### Example 2: Degradation of Intracellular mCherry Protein by ARRDC1/sdAb Fusion Protein Delivered by ARMMs

This example demonstrates a decrease in intracellular mCherry protein levels by incubation of target cells with ARMMs particles payloaded with an ARRDC1 fusion protein comprising an sdAb specific to mCherry protein.

ARMMs particles payloaded with ARRDC1/GFP fusion protein or ARRDC1/anti-mCherry sdAb fusion protein were produced by culturing suspension-adapted cell line derived from HEK293 cells (ThermoFisher) that were stably transfected with expression constructs encoding the relevant fusion protein. Additionally, “pseudotyped” ARRDC1/anti-mCherry sdAb fusion protein payloaded ARMMs were produced by cells that had been engineered to overexpress vesicular stomatitis virus G protein (VSV-G). ARMMs particles were produced by culturing the relevant cell lines in shaker flasks, collecting conditioned media, collecting, concentrating and purifying the ARMMs particles by centrifugation to remove cellular debris, followed by ultafiltration/diafiltration (UF/DF) using tranverse flow filtration (TFF) using a 50 nm pore hollow fiber filtration cassette.

Adherent HEK293T target cells were plated, transfected with 50 ng mCherry expression construct as described in Example 1, cultured for 1 day, then reseeded. 1×10⁶ ARMMs per target cell were added approximately 6 hours later, and the target cells were cultured for 2 days. An additional 1×10⁶ ARMMs per target cell were added to the cultures, and the cells were cultured for 2 additional days, followed by harvest of the cells and preparation of whole cell extracts for Western blotting. One set of negative control target cells were treated with vehicle (phosphate-buffered saline, PBS), and a second set of negative control target cells were untreated.

Western blots were stained with anti-GAPDH (gel/blot loading control), ant-ARRDC1, and anti-mCherry. As shown in FIG. 8, target cell mCherry levels were unaffected by negative controls (lanes 1 and 5), but reduced by ARMMs-delivered ARRDC1/anti-mCherry sdAb fusion protein, particularly the VSV-G pseudotyped ARMMs particles.

What is claimed is:

1. An arrestin domain-containing protein 1 (ARRDC1) fusion protein comprising:
an ARRDC1 protein or a variant thereof, and a non-enzymatic targeting domain that recruits a target protein.

2. The ARRDC1 fusion protein of claim 1, wherein said targeting domain is fused to the C-terminal end of said ARRDC1 protein or a variant thereof.

3. The ARRDC1 fusion protein of any one of claims 1-2, wherein said ARRDC1 protein or a variant thereof is fused to said targeting domain by a linker.

4. The ARRDC1 fusion protein of any one of claims 1-3, wherein said ARRDC1 protein or a variant thereof comprises at least two PPXY motifs.

5. The ARRDC1 fusion protein of claim 4, wherein said at least two PPXY motifs are different.

6. The ARRDC1 fusion protein of any one of claims 1-5, wherein said non-enzymatic targeting domain comprises an interaction domain.

7. The ARRDC 1 fusion protein of claim 6, wherein said interaction domain comprises a non-enzymatic protein binding peptide.

8. The ARRDC1 fusion protein of any one of claims 1-7, wherein said target protein is a transcription factor, a tumor promoter or a protein product of an oncogene, a developmental regulator, a growth factor, a metastasis promotor, an anti-apoptotic protein, a membrane-associated protein, a transmembrane receptor, an enzyme, a nuclease, a recombinase, or a reprogramming factor.

9. The ARRDC1 fusion protein of any one of claims 1-8, wherein said non-enzymatic targeting domain comprises an intrabody.

10. The ARRDC1 fusion protein of claim 9, wherein said intrabody comprises a single domain antibody (sdAb).

11. The ARRDC1 fusion protein of claim 10, wherein said sdAb is a camelid single domain.

12. The ARRDC1 fusion protein of any one of claims 1-11, wherein a terminal of said non-enzymatic targeting domain is fused to at least one additional PPXY motif.

13. The ARRDC1 fusion protein of claim 12, wherein the terminal of said non-enzymatic targeting domain is fused to at least two additional PPXY motifs.

14. The ARRDC1 fusion protein of claim 13, wherein said at least two additional PPXY motifs are different PPXY motifs.

15. The ARRDC1 fusion protein of any one of claims 1-14, wherein said non-enzymatic targeting domain is fused to a Fc region of an antibody.

16. The ARRDC1 fusion protein of claim 15, wherein said non-enzymatic targeting domain or said Fc region of an antibody is fused to an RNA binding protein.

17. The ARRDC1 fusion protein of claim 16, wherein said RNA binding protein is a tat protein.

18. The ARRDC1 fusion protein of claim 17, whrein said tat protein is an HIV tat protein or a BIV tat protein.

19. The ARRDC1 fusion protein of claim 1, wherein said targeting domain comprises a bound aptamer.

20. The ARRDC1 fusion protein of any one of claims 1-19, wherein said targeting domain is fused to a ubiquitin ligase.

21. The ARRDC1 fusion protein of any one of claim 3-19, wherein said linker is a cleavable linker.

22. The ARRDC1 fusion protein of claim 20, wherein said cleavable linker comprises a protease cleavage site.

23. The ARRDC1 fusion protein of claim 22, wherein said protease cleavage site is a furin cleavage site.

24. The ARRDC1 fusion protein of claim 22, wherein said protease cleavage site is a viral cleavage site.

25. The ARRDC1 fusion protein of claim 23, wherein said viral cleavage site comprises an HIV-1 protease cleavage site, a tobacco etch virus (TEV) cleavage site, or another cleavage site recognized by a viral protease.

26. The ARRDC1 fusion protein of claim 21, wherein said cleavable linker is a photocleavable linker that comprises a photocleavable protein or fragment thereof.

27. The ARRDC1 fusion protein of any one of claims 1-26, wherein said photocleavable linker comprises a photocleavable protein PhoCll or a fragment thereof.

28. The ARRDC1 fusion protein of claim any one of claims 26-27, wherein said photocleavable linker comprises a protein or protein fragment derived from PhoCl1.

29. The ARRDC1 fusion protein of claim 20, wherein said ubiquitin ligase is a ubiquibody.

30. The ARRDC1 fusion protein of claim 29, wherein said ubiquibody is CHIP (carboxyl terminus of Hsc70-interacting protein) or a fragment thereof.

31. The ARRDC1 fusion protein of claim 1, wherein said non-enzymatic targeting domain comprises a ubiquitination-specific antibody domain.

32. The ARRDC1 fusion protein of any one of claims 1-31, wherein said ARRDC1 protein or said variant thereof comprises a FLAG-tag.

33. The ARRDC1 fusion protein of claim 32, wherein said FLAG-tag comprises a sequence of DYKDDDK (SEQ ID NO: 90).

34. An arrestin domain-containing protein 1 (ARRDC1)-mediated microvesicle (ARMM), comprising:
(i) a lipid bilayer; and
(ii) the ARRDC1 fusion protein or a variant thereof of any of claims 1-33.

35. The ARMM of claim 34, further comprising a fusion RNA, wherein said fusion RNA comprises (i) a binding RNA sequence that binds said RNA binding protein and (ii) an expression sequence encoding a ubiquitin ligase.

36. The ARMM of claim 35, wherein said RNA sequence is a TAR sequence.

37. The ARMM of claim 35 or 36, wherein said ubiquitin ligase is Trim21.

38. A nucleic acid construct encoding the ARRDC1 fusion protein of any of claims 1-33.

39. A microvesicle-producing cell, comprising a recombinant expression construct encoding the ARRDC1 fusion protein of any of claims 1-33 under the control of a heterologous promoter.

40. The microvesicle-producing cell of claim 39, further comprising a recombinant expression construct encoding a fusion RNA under the control of a heterologous promoter, wherein said fusion RNA comprises (i) a binding RNA sequence that binds said RNA binding protein and (ii) an expression sequence encoding a ubiquitin ligase.

41. The microvesicle-producing cell of claim 40, wherein said RNA sequence is a TAR sequence.

42. The microvesicle-producing cell of claim 41, wherein said ubiquitin ligase is Trim21.

43. A method for degrading a target protein, comprising contacting the target cell with the microvesicle of any one of claims 34-37.

44. A method for altering expression of at least one gene, comprising contacting the target cell with the microvesicle of any one of claims 34-37.

45. A method for preparing an arrestin domain-containing protein 1 (ARRDC1)-mediated microvesicle (ARMM), comprising
(a) culturing a host cell comprising a recombinant expression construct encoding the ARRDC1 fusion protein of any one of claims 1-33 under the control of a heterologous promoter in a medium; and
(b) collecting said ARMM from said host cell or said medium.

46. The method of claim 45, wherein said host cell has a reduced expression level of ubiquitin ligase as compared to a host cell of wildtype.

47. The method of claim 46, wherein said host cell lacks said ubiquitin ligase.

48. The method of any one of claims 45-47, wherein said host cell has a reduced expression level of said target protein as compared to a host cell of wildtype.

49. The method of claim 48, wherein said host cell lacks said target protein.


SEQUENCE LISTING (109 sequences)

SEQ ID NO: 1 - protein, 4 aa, Homo sapiens
PSAP

SEQ ID NO: 2 - skipped

SEQ ID NO: 3 - protein, 433 aa, Homo sapiens
MGRVQLFEISLSHGRVVYSPGEPLAGTVRVRLGAPLPFRAIRVTCIGSCGVSNKANDTAWVVEEGYFNSSLSLADKGSLPAGEHSFPFQFLLPATAPTSFEGPFGKIVHQVRAAIHTPRFSKDHKCSLVFYILSPLNLNSIPDIEQPNVASATKKFSYKLVKTGSVVLTASTDLRGYVVGQALQLHADVENQSGKDTSPVVASLLQKVSYKAKRWIHDVRTIAEVEGAGVKAWRRAQWHEQILVPALPQSALPGCSLIHIDYYLQVSLKAPEATVTLPVFIGNIAVNHAPVSPRPGLGLPPGAPPLVVPSAPPQEEAEAEAAAGGPHFLDPVFLSTKSHSQRQPLLATLSSVPGAPEPCPQDGSPASHPLHPPLCISTGATVPYFAEGSGGPVPTTSTLILPPEYSSWGYPYEAPPSYEQSCGGVEPSLTPES

SEQ ID NO: 4 - protein, 434 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQKVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 5 - protein, 433 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 6 - protein, 101 aa, synthetic construct
MEPVDPRLEPWKHPGSQPRTPCTTCYCKKCCFHCQVCFTTKALGISYGRKKRRQRRRPPQGSQTHQVSLSKQPSSQPRGDQTGPKESKKKVERETEADPKP

SEQ ID NO: 7 - protein, 86 aa, synthetic construct
MEPVDPRLEPWKHPGSQPRTPCTTCYCKKCCFHCQVCFTTKALGISYGRKKRRQRRRPPQGSQTHQVSLSKQPSSQPRGDQTGPKE

SEQ ID NO: 8 - protein, 36 aa, synthetic construct
CFTTKALGISYGRKKRRQRRRPPQGSQTHQVSLSKQ

SEQ ID NO: 9 - protein, 45 aa, synthetic construct
MEPVDPRLEPWKHPGSQPRTPCTTCYCKKCCFHCQVCFTTKALGI

SEQ ID NO: 10 - protein, 38 aa, synthetic construct
RKKRRQRRRPPQGSQTHQVSLSKQPSSQPRGDQTGPKE

SEQ ID NO: 11 - protein, 35 aa, synthetic construct
RRQRRRPPQGSQTHQVSLSKQPSSQPRGDQTGPKE

SEQ ID NO: 12 - protein, 32 aa, synthetic construct
RRRPPQGSQTHQVSLSKQPSSQPRGDQTGPKE

SEQ ID NO: 13 - protein, 29 aa, synthetic construct
PPQGSQTHQVSLSKQPSSQPRGDQTGPKE

SEQ ID NO: 14 - protein, 9 aa, synthetic construct
RKKRRQRRR

SEQ ID NO: 15 - protein, 11 aa, synthetic construct
RKKRRQRRRPP

SEQ ID NO: 16 - protein, 13 aa, synthetic construct
RKKRRQRRRPPQG

SEQ ID NO: 17 - protein, 15 aa, synthetic construct
RKKRRQRRRPPQGSQ

SEQ ID NO: 18 - protein, 17 aa, synthetic construct
RKKRRQRRRPPQGSQTH

SEQ ID NO: 19 - protein, 21 aa, synthetic construct
CFTTKALGISYGRKKRRQRRR

SEQ ID NO: 20 - protein, 27 aa, synthetic construct
CFTTKALGISYGRKKRRQRRRPPQGSQ

SEQ ID NO: 21 - protein, 12 aa, synthetic construct
GRRKKRRQRRRP

SEQ ID NO: 22 - protein, 17 aa, synthetic construct
RKKRRQRRRPPQGSQTH

SEQ ID NO: 23 - protein, 130 aa, synthetic construct
METPLKAPEGSLGSYNEPSSCTSEQDAAAQGLVSPGDEILYQLYQPLEACDNKCYCKKCCYHCQMCFLNKGLGIWYERKGRRRRTPKKTKAHSSSASDKSISTRTGNSQPEKKQKKTLETALETIGGPGR

SEQ ID NO: 24 - protein, 103 aa, synthetic construct
MPGPWVAMIMLPQPKESFGGKPIGWLFWNTCKGPRRDCPHCCCPICSWHCQLCFLQKNLGINYGSGPRRRGTRGKGRRIRRTASGGDQRREADSQRSFTNMDQ

SEQ ID NO: 25 - protein, 17 aa, synthetic construct
SGPRPRGTRGKGRRIRR

SEQ ID NO: 26 - RNA, 34 nt, synthetic construct
  source 1..34: mol_type = other RNA
ggtctgggcgcagcgcaagctgacggtacaggcc

SEQ ID NO: 27 - RNA, 35 nt, synthetic construct
  source 1..35: mol_type = other RNA
ggctggactcgtacttcggtactggagaaacagcc

SEQ ID NO: 28 - protein, 116 aa, synthetic construct
MAGRSGDSDEELIRTVRLIKLLYQSNPPPNPEGTRQARRNRRRRWRERQRQIHSISERILGTYLGRSAEPVPLQLPPLERLTLDCNEDCGTSGTQGVGSPQILVESPTVLESGTKE

SEQ ID NO: 29 - protein, 17 aa, synthetic construct
TRQARRNRRRRWRERQR

SEQ ID NO: 30 - protein, 23 aa, synthetic construct
RDRRRRGSRPSGAERRRRRAAAA

SEQ ID NO: 31 - RNA, 19 nt, synthetic construct
  source 1..19: mol_type = other RNA
acatgaggattacccatgt

SEQ ID NO: 32 - RNA, 18 nt, synthetic construct
  source 1..18: mol_type = other RNA
ccggaggatcaccacggg

SEQ ID NO: 33 - RNA, 14 nt, synthetic construct
  source 1..14: mol_type = other RNA
ccacagtcactggg

SEQ ID NO: 34 - protein, 129 aa, synthetic construct
ASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 35 - protein, 20 aa, synthetic construct
NAKTRRHERRRKLAIERDTI

SEQ ID NO: 36 - protein, 24 aa, synthetic construct
GSMDAQTRRRERRAEKQAQWKAAN

SEQ ID NO: 37 - protein, 19 aa, synthetic construct
GTAKSRYKARRAELIAERR

SEQ ID NO: 38 - protein, 55 aa, synthetic construct
MQKGNFRNQRKTVKCFNCGKEGHIAKNCRAPRKKGCWKCGKEGHQMKDCTERQAN

SEQ ID NO: 39 - RNA, 63 nt, synthetic construct
  source 1..63: mol_type = other RNA
gggtctctctggttagaccagatctgagcctgggagctctctggctaactagggaacccactg

SEQ ID NO: 40 - RNA, 59 nt, synthetic construct
  source 1..59: mol_type = other RNA
gggtctctctggttagaccagatctgagcctgggctctggctaactagggaacccactg

SEQ ID NO: 41 - RNA, 58 nt, synthetic construct
  source 1..58: mol_type = other RNA
gggtctctctggttagaccagatctgagcctgggagctctctggctaactagggaacc

SEQ ID NO: 42 - RNA, 23 nt, synthetic construct
  source 1..23: mol_type = other RNA
agatctgagcctgggagctctct

SEQ ID NO: 43 - RNA, 28 nt, synthetic construct
  source 1..28: mol_type = other RNA
gctcgttgagctctgggaagctccgagc

SEQ ID NO: 44 - RNA, 22 nt, synthetic construct
  source 1..22: mol_type = other RNA
tcgtgtagctcattagctccga

SEQ ID NO: 45 - RNA, 15 nt, synthetic construct
  source 1..15: mol_type = other RNA
gcgctgacaaagcgc

SEQ ID NO: 46 - RNA, 19 nt, synthetic construct
  source 1..19: mol_type = other RNA
gggccctgaagaagggccc

SEQ ID NO: 47 - RNA, 20 nt, synthetic construct
  source 1..20: mol_type = other RNA
tctcaacctaaccgttgaga

SEQ ID NO: 48 - RNA, 20 nt, synthetic construct
  source 1..20: mol_type = other RNA
ggactagcggaggctagtcc

SEQ ID NO: 49 - protein, 5 aa, synthetic construct
GGGGS

SEQ ID NO: 50 - protein, 10 aa, synthetic construct
GGGGSGGGGS

SEQ ID NO: 51 - protein, 15 aa, synthetic construct
GGGGSGGGGSGGGGS

SEQ ID NO: 52 - protein, 20 aa, synthetic construct
GGGGSGGGGSGGGGSGGGGS

SEQ ID NO: 53 - protein, 8 aa, synthetic construct
GGGGGGGG

SEQ ID NO: 54 - protein, 6 aa, synthetic construct
GGGGGG

SEQ ID NO: 55 - protein, 5 aa, synthetic construct
EAAAK

SEQ ID NO: 56 - protein, 10 aa, synthetic construct
EAAAKEAAAK

SEQ ID NO: 57 - protein, 15 aa, synthetic construct
EAAAKEAAAKEAAAK

SEQ ID NO: 58 - protein, 46 aa, synthetic construct
AEAAAKEAAAKEAAAKEAAAKALEAEAAAKEAAAKEAAAKEAAAKA

SEQ ID NO: 59 - protein, 5 aa, synthetic construct
PAPAP

SEQ ID NO: 60 - protein, 12 aa, synthetic construct
AEAAAKEAAAKA

SEQ ID NO: 61 - skipped

SEQ ID NO: 62 - protein, 17 aa, synthetic construct
VSQTSKLTRAETVFPDV

SEQ ID NO: 63 - protein, 6 aa, synthetic construct
PLGLWA

SEQ ID NO: 64 - protein, 6 aa, synthetic construct
RVLAEA

SEQ ID NO: 65 - protein, 10 aa, synthetic construct
EDVVCCSMSY

SEQ ID NO: 66 - protein, 8 aa, synthetic construct
GGIEGRGS

SEQ ID NO: 67 - protein, 10 aa, synthetic construct
TRHRQPRGWE

SEQ ID NO: 68 - protein, 10 aa, synthetic construct
AGNRVRRSVG

SEQ ID NO: 69 - protein, 9 aa, synthetic construct
RRRRRRRRR

SEQ ID NO: 70 - protein, 4 aa, synthetic construct
GFLG

SEQ ID NO: 71 - skipped

SEQ ID NO: 72 - protein, 18 aa, synthetic construct
KESGSVSSEQLAQFRSLD

SEQ ID NO: 73 - protein, 14 aa, synthetic construct
EGKSSGSGSESKST

SEQ ID NO: 74 - protein, 12 aa, synthetic construct
GSAGSAAGSGEF

SEQ ID NO: 75 - skipped

SEQ ID NO: 76 - protein, 20 aa, synthetic construct
LEAGCKNFFPRSFTSCGSLE

SEQ ID NO: 77 - protein, 12 aa, synthetic construct
CRRRRRREAEAC

SEQ ID NO: 78 - skipped

SEQ ID NO: 79 - protein, 12 aa, synthetic construct
TRHRQPRGWEQL

SEQ ID NO: 80 - protein, 9 aa, synthetic construct
RRRRRRRRR

SEQ ID NO: 81 - protein, 10 aa, synthetic construct
TRHRQPRGWE

SEQ ID NO: 82 - protein, 10 aa, synthetic construct
AGNRVRRSVG

SEQ ID NO: 83 - protein, 11 aa, synthetic construct
RHRQPRGWEQL

SEQ ID NO: 84 - protein, 7 aa, synthetic construct
SQNYIVQ

SEQ ID NO: 85 - protein, 11 aa, synthetic construct
NFSQNYIVQTG

SEQ ID NO: 86 - protein, 6 aa, synthetic construct
ENLYFQ

SEQ ID NO: 87 - protein, 6 aa, synthetic construct
ENLYFQ

SEQ ID NO: 88 - protein, 36 aa, synthetic construct
GGGGSGGGGSGGGGSENLYFQGGGGSGGGGSGGGGS

SEQ ID NO: 89 - protein, 36 aa, synthetic construct; identical to SEQ ID NO: 88

SEQ ID NO: 90 - protein, 7 aa, synthetic construct
DYKDDDK

SEQ ID NO: 91 - protein, 12 aa, synthetic construct
APAPAPAPAPAP

SEQ ID NO: 92 - protein, 14 aa, synthetic construct
APAPAPAPAPAPAP

SEQ ID NO: 93 - protein, 16 aa, synthetic construct
APAPAPAPAPAPAPAP

SEQ ID NO: 94 - protein, 18 aa, synthetic construct
APAPAPAPAPAPAPAPAP

SEQ ID NO: 95 - protein, 20 aa, synthetic construct
APAPAPAPAPAPAPAPAPAP

SEQ ID NO: 96 - protein, 22 aa, synthetic construct
APAPAPAPAPAPAPAPAPAPAP

SEQ ID NO: 97 - protein, 24 aa, synthetic construct
APAPAPAPAPAPAPAPAPAPAPAP

SEQ ID NO: 98 - protein, 26 aa, synthetic construct
APAPAPAPAPAPAPAPAPAPAPAPAP

SEQ ID NO: 99 - protein, 28 aa, synthetic construct
APAPAPAPAPAPAPAPAPAPAPAPAPAP

SEQ ID NO: 100 - protein, 30 aa, synthetic construct
APAPAPAPAPAPAPAPAPAPAPAPAPAPAP

SEQ ID NO: 101 - protein, 32 aa, synthetic construct
APAPAPAPAPAPAPAPAPAPAPAPAPAPAPAP

SEQ ID NO: 102 - protein, 34 aa, synthetic construct
APAPAPAPAPAPAPAPAPAPAPAPAPAPAPAPAP

SEQ ID NO: 103 - skipped

SEQ ID NO: 104 - protein, 7 aa, synthetic construct
SQNPIVQ

SEQ ID NO: 105 - protein, 11 aa, synthetic construct
NFSQNPIVQTG

SEQ ID NO: 106 - protein, 6 aa, synthetic construct
ENLYFG

SEQ ID NO: 107 - protein, 6 aa, synthetic construct
ENLYFS

SEQ ID NO: 108 - protein, 36 aa, synthetic construct
GGGGSGGGGSGGGGSENLYFGGGGGSGGGGSGGGGS

SEQ ID NO: 109 - protein, 36 aa, synthetic construct
GGGGSGGGGSGGGGSENLYFSGGGGSGGGGSGGGGS